(19) United States
(12) Patent Application Publication
(10) Pub. No.: US 2021/0230608 A1
(43) Pub. Date: Jul. 29, 2021

(54) GYCOMODULE MOTIFS AND USES THEREOF

(71) Applicant: GAT BIOSCIENCES, S.L., Barcelona (ES)

(72) Inventors: Olga DURANY TURK, Barcelona (ES); Jordi SEGURA DE YEBRA, Barcelona (ES); Jaume MERCADE ROCA, Barcelona (ES); Maria Teresa LOPEZ CERRO, Barcelona (ES); Cristina LOPEZ PAZ, Barcelona (ES)

(21) Appl. No.: 17/053,525

(22) PCT Filed: May 9, 2019
(86) PCT No.: PCT/EP2019/061916

§ 371 Date: Nov. 6, 2020

(30) Foreign Application Priority Data
May 9, 2018 (EP) 18382319.4

(51) Int. Cl. C12N 15/79; C12P 21/00

(52) U.S. Cl. CPC C12N 15/79; C12P 21/005

(57) ABSTRACT

A nucleotide sequence encoding a glycomodule motif having a sequence selected from SEQ ID NO 1, SEQ ID NO 2, SEQ ID NO 3, SEQ ID NO 4, SEQ ID NO 5 and a functionally equivalent variant thereof is provided. Also provided are an expression cassette having at least one nucleotide sequence encoding a glycomodule motif, a vector and host cell and a method for expressing a protein of interest.

### TECHNICAL FIELD OF THE INVENTION

The present invention relates to a method for improving recombinant protein production in microalgae.

### BACKGROUND OF THE INVENTION

Recombinant protein (RP) production has an enormous economic importance due to its application on therapy, diagnostic and industry. Most common host organisms for the production of recombinant proteins are microorganisms (yeast and bacteria). Among bacteria, E. coli is by far the most commonly used organism, however, its use is limited to small and non-complex proteins that do not require complex posttranslational modifications. Another disadvantage of using bacteria as a host for RP production is the presence of endotoxins and possible pathogens in the final product.

Yeast is an alternative for the production of RP because it allows the synthesis of complex proteins in an organism that has the advantage of a low cost production. A different protein glycosylation pattern that usually involves hyperglycosylation, different from what occurs in higher organisms may become a major limitation for certain uses of RPs produced in yeast.

Mammalian cells are also routinely used for the production of recombinant proteins but high costs of production together with biosafety requirements limits its use to proteins of therapeutic interest.

Transgenic plants present advantages as RP biofactories: low production cost, free of endotoxin and viral agents plus ease to scale up. Drawbacks of plants as bio factories are slow growth, time required for transgenic generation, the possibility of gene flow when grown without containment plus an expensive downstream purification.

Microalgae as protein factories have gained attention in the last years, due to increased knowledge and availability of new molecular genetic tools. Microalgae share with plants advantages as producers of recombinant proteins in terms of production, scale up and safety, but in addition, they can be cultivated in contained reactors in minimal media, therefore deleting risks of environmental contamination. Importantly, the time required for production of RP is also shorter than is the case of transgenic plants. Since microalgae grow as cell cultures, proteins can be secreted to the media, which is basically a salt containing media, low in protein content and impurities that may add immunological reactions. Many species of microalgae are considered GRAS (generally regarded as safe) which is an additional advantage for certain uses of RP.

Among microalgae, Chlamydomonas is the most extensively studied organism. It has been used as model organism for the study of different processes such as photosynthesis, cell cycle, flagelar study, or light perception for more than 60 years. More recently it has gained attention as a platform for RP production. Chlamydomonas has unique advantages to be a reference organism in biotechnology including methods for genetic transformation of all three genomes, a high growth rate, low growth cost, ease of cultivation and ability to secrete proteins to the media

Despite its potential as a host for the production RP a major limitation step are the low titers of RP obtained from nuclear expression. Positional effects due to random integration of the transgene in the genome, a potent silencing machinery and limited research compared to other most commonly studied organisms, may account for the reasons of low heterologous expression.

To overcome Chlamydomonas poor transgene expression several independent strategies have been successfully used. Codon optimization (Ruecker, et al. Mol. Genet. Genomics. 2008. 280: 153-162, Barahimipour, R. et al. Plant J. 2015. 84: 704-17), use of introns in sequences (Lumbreras, V. et al. EMBO Rep. 2001. 2: 55-60, Sizova, I. et al. Gene. 2001. 277: 221-229, Hu, J., et al. Plant J. 2014. 79: 1052-64), use of endogenous robust promoters and UTRS (Schroda, M., et al. Plant J. 2002. 31: 445-455; Fischer, N. & Rochaix, J. D. Mol Genet Genomics. 2001. 265: 888-894; López-Paz et al. Plant J. 2017. 96: 1232-1244), secretory peptides (Lauersen, K. et al. J. Biotechnol. 2013.167: 101-110; Molino, J. V. D., et al. PLoS One. 2013, e0192433), fusion to reporter genes (Fuhrmann, M., Oertel, W. & Hegemann, P. Plant J. 1999. 19: 353-361, EP2684960 A1), obtention of high producing strains (Barahimipour, R., et al. Plant Mol. Biol. 2017. 90: 403-418) or more recently, use of glycomodules that confer stability to recombinant secreted proteins are all examples of strategies to improve transgene expression (Ramos-Martinez, E. M., et al. Plant Biotechnol. J. 2017. 15: 1214-1224). The use of glycomodules to enhance protein secretion and accumulation has been described both in plant cells and Chlamydomonas (U.S. Pat. No. 9,006,410B2, EP1711533B1). For instance, (SP)₁₀ and (SP)₂₀ synthetic glycomodules were introduced in a recombinant protein resulting in a yield up to 12 fold the yield of protein without glycomodules (Ramos-Martinez, E. M., et al. Plant Biotechnol. J. 15, 1214-1224 (2017)).

The complex nature of the mechanisms leading to recombinant protein expression needs designing new strategies that allow for protein secretion and purification improvement.

### SUMMARY OF THE INVENTION

The authors of the present invention have identified the presence of glycomodule motifs (GM) in some of the most abundant Chlamydomonas secreted proteins. The identified sequences confer increased stability when fused to recombinant proteins. Here, it is described the use of a DNA vector containing different glycomodules in combination with other elements to successfully improve Chlamydomonas recombinant protein secretion and accumulation of recombinant protein in the media.

In a first aspect, the invention relates to a nucleotide sequence encoding a glycomodule motif having a sequence selected from the group consisting of SEQ ID NO 1, SEQ ID NO 2, SEQ ID NO 3, SEQ ID NO 4, SEQ ID NO 5 and a functionally equivalent variant thereof.

In a second aspect, the invention relates to an expression cassette comprising at least one nucleotide sequence encoding a glycomodule motif according to claim 1.

In a third aspect, the invention relates to a vector comprising a nucleotide sequence encoding a glycomodule motif according to the invention or an expression cassette according to the invention.

In a fourth aspect, the invention relates to a host cell comprising a vector of the invention.

In a fifth aspect, the invention relates to a method for expressing a protein of interest which comprises growing a microalga cell comprising a vector according to the invention, wherein the vector comprises a nucleotide sequence encoding a protein of interest and growing said cell in conditions suitable for allowing the expression of the protein of interest.

In a sixth aspect, the invention relates to the use of a nucleotide sequence encoding a glycoprotein motif according to the invention, an expression cassette according to the invention, a vector according to the invention or a host cell according to the invention for the expression of a protein of interest.

### BRIEF DESCRIPTION OF THE FIGURES

FIG. 1. Schematics of proposed gene cassettes used to improve transgene expression in Chlamydomonas. ARSss: signal sequence from ARS (Chlamydomonas reinhardtii periplasmic arylsulfatase), 6×His: Histidine Tag; I: Intron May be intron from RBCS2 or any other highly expressed gene; SP: (SP)ₙ synthetic glycomodule; glycomodule sequences derived from Chlamydomonas most abundant secreted proteins are named according to original protein from where they were identified: LCL, GP1, GP2, PHC21. TEV: Protease recognition sequence may be TEV protease or any other specific protease. Protein of interest is hEGF: Human epidermal growth factor. Reporter protein used is gLuc (Gaussia princess luciferase).

FIG. 2. Comparison of luciferase expression in transformants containing different gene cassettes. Distribution of normalized luciferase expression (RLU) values from Chlamydomonas reinhardtii (A) CC1-24 or (B) UVM4 transformed with constructs illustrated in FIG. 2. 48 independent transformants (A) or 96 independent transformants (B) were analyzed for each construct. * indicates a significant amount of highly expressing transformants compared to parsLuc-EGF transformants (Mann-Whitney U test, p<0.05)

FIG. 3. Immunoblot analysis of transformants expressing different Luc:EGF gene cassettes described in FIG. 2. Clones with the highest expression as determined by luciferase assay were selected for this analysis. Equal amounts of concentrated media from equally grown cells were loaded on each lane. MW: Molecular Weight protein marker; GLuc: purified recombinant gLuciferase protein produced in E. coli was used as a control.

FIG. 4. Western blot quantification of different secreted fusion proteins. Different amounts of concentrated media were loaded. A positive GLuc recombinant protein of known concentration is used as a control of quantification. Primary antibody: rabbit polyclonal anti GLuc.

FIG. 5. IMAC purification of RP secreted proteins. An immunoblot against Gluc was performed to determine efficiency of recovery of protein from the media before and after digestion with TEV Protease. Briefly, Concentrated media (I:Input) from highly expressing transformants (parsLucEGF, parsLucEGF-SP10 and parsLucEGF-SP20) was incubated with a nickel agarose resin. FT: Flow through, represents not bound protein. Eluted fractions are treated with TEV protease and a second IMAC is performed after digestion. Fusion proteins are completely digested and only Gluc remains bound to the resin. Elution and FT are submitted to dialysis and concentration and E1-1, E1-2, E1-3, FT-1 and FT-2 are samples of this intermediate steps.

### DETAILED DESCRIPTION OF THE INVENTION

### Glycomodule Motifs

In a first aspect the invention relates to a nucleotide sequence encoding a glycomodule motif having a sequence selected from the group consisting of SEQ ID NO 1, SEQ ID NO 2, SEQ ID NO 3, SEQ ID NO 4, SEQ ID NO 5 and a functionally equivalent variant thereof.

The term “nucleotide sequence”, as used herein refers to a single-stranded or double-stranded sequence having deoxyribonucleotide (DNA) or ribonucleotide (RNA) bases. In a preferred embodiment the nucleotide sequence is RNA. In a more preferred embodiment the nucleotide sequence is DNA.

“A glycomodule motif (GM)”, as used herein, refers to an amino acid sequence comprising at least one residue that can be either hydroxylated and glycosylated or a residue that can be glycosylated. As used herein, the term “glycosylation site” is meant to refer an amino acid that acts as a target site of glycosylation. In a preferred embodiment, the glycosylation site is an amino acid sequence that acts as a target for glycosylation in a microalga. Glycosylation is the reaction catalysed by glycosyltransferases, which adds carbohydrates site-specifically to another molecule, preferably proteins. Glycosylation of proteins may come in different forms, such as N-linked, O-linked and phosphoserine glycosylation. Non-limiting examples of amino acids that can become glycosylated include: proline, serine, threonine, hydroxylysine, hydroxyproline, arginine and asparagine. Thus, within glycosylation sites, proline residues may be hydroxylated to form hydroxyprolines (Hyp). In a preferred embodiment glycosylation takes place in any serine (Ser) or hydroxiproline (Pro) of the glycomodule motif. The sites for glycosylation can be placed at either or both termini of the glycomodule motif, and/or in the interior of the glycomodule if desired. Preferably, the glycosylation of the glycomodule motifs of the invention is O-Glycosylation.

Hydroxyproline O-Glycosylation is generally of two types: 1) arabinogalactan glycomodules comprise clustered non-contiguous hydroxyproline (Hyp) residues in which the Hyp residues are O-glycosylated with arabinogalactan adducts; and 2) arabinosylation glycomodules comprise contiguous Hyp residues in which some or all of the Hyp residues are arabinosylated (O-glycosylated) with chains of arabinose about 1-5 residues long. O-Glycosylation may occur following hydroxylation of the one or more of the residues in the site.

SEQ ID NO: 1 as disclosed herein relates to a glycomodule motif derived from the protein LCL5 of sequence shown in SEQ ID NO: 6.

SEQ ID NO: 2 as disclosed herein relates to a glycomodule motif derived from the protein GP1 of sequence SEQ ID NO: 7.

SEQ ID NO: 3 as disclosed herein relates to a glycomodule motif derived from the protein GP2 of sequence SEQ ID NO: 8.

SEQ ID NO: 4 as disclosed herein relates to a glycomodule motif derived from the protein PHC21 of sequence SEQ ID NO: 9.

SEQ ID NO: 5 as disclosed herein relates to a glycomodule motif derived from the protein PHC21 of sequence SEQ ID NO: 9.

“Functionally equivalent variant of SEQ ID NO: 1, SEQ ID NO: 2, SEQ ID NO: 3, SEQ ID NO: 4 or SEQ ID NO: 5”, as used herein, relates to all those sequences which result from the modification, insertion and/or deletion of one or more amino acids from the above sequence, provided that the function of the glycomodule motif is substantially maintained, particularly, the increased yield and secretion of a protein comprising a glycomodule motif variant of the invention and excluding the whole protein LCL5 (SEQ ID NO: 6), GP1 (SEQ ID NO: 7), GP2 (SEQ ID NO: 8) and PHC21 (SEQ ID NO: 9).

Suitable assays for determining whether a polypeptide can be considered as a functionally equivalent variant of the glycomodules of the invention include, without limitation: staining of glycoproteins (e.g. methods based on Periodic acid Schiff stain), enzymatic or chemical removal of glycomodules and analysis by Western blot and/or Mass Spectrometry.

Preferably, variants of SEQ ID NO: 1, SEQ ID NO: 2, SEQ ID NO: 3, SEQ ID NO: 4 or SEQ ID NO: 5 are (i) polypeptides in which one or more amino acid residues are substituted by a preserved or non-preserved amino acid residue (preferably a preserved amino acid residue) and such substituted amino acid may be coded or not by the genetic code, (ii) polypeptides in which there is one or more modified amino acid residues, for example, residues modified by substituent bonding, (iii) polypeptides resulting from alternative processing of a similar mRNA, (iv) polypeptide fragments and/or (v) polypeptides resulting from fusion of the polypeptide defined in (i) to (iii) with another polypeptide, such as a secretory leader sequence or a sequence being used for purification (for example, His tag) or for detection (for example, Sv5 epitope tag). The fragments include polypeptides generated through proteolytic cut (including multisite proteolysis) of an original sequence. The variants may be post-translationally or chemically modified. Such variants are supposed to be apparent to those skilled in the art.

One skilled in the art will recognize that the values of identity of nucleotide sequences can be appropriately adjusted in order to determine the corresponding sequence identity of two nucleotide sequences encoding the polypeptides of the present invention, by taking into account codon degeneracy, conservative amino acid substitutions, and reading frame positioning.

In the context of the present invention “conservative amino acid changes” and “conservative amino acid substitution” are used synonymously in the invention. “Conservative amino acid substitutions” refers to the interchangeability of residues having similar side chains, and mean substitutions of one or more amino acids in a native amino acid sequence with another amino acid(s) having similar side chains, resulting in a silent change that does not alter function of the protein. Conserved substitutes for an amino acid within a native amino acid sequence can be selected from other members of the group to which the naturally occurring amino acid belongs. For example, a group of amino acids having aliphatic side chains includes glycine, alanine, valine, leucine, and isoleucine; a group of amino acids having aliphatic-hydroxyl side chains includes serine and threonine; a group of amino acids having amide-containing side chains includes asparagine and glutamine; a group of amino acids having aromatic side chains includes phenylalanine, tyrosine, and tryptophan; a group of amino acids having basic side chains includes lysine, arginine, and histidine; and a group of amino acids having sulfur-containing side chains includes cysteine and methionine. In some embodiments of the invention, preferred conservative amino acids substitutions are: valine-leucine, valine-isoleucine, phenylalanine-tyrosine, lysine-arginine, alanine-valine, aspartic acid-glutamic acid, and asparagine-glutamine. Thus, the invention refers to functionally equivalents variants of SEQ ID NO: 1, SEQ ID NO: 2, SEQ ID NO: 3, SEQ ID NO: 4 or SEQ ID NO: 5; and that have an amino acid sequence differing in one or more amino acids with the sequence given as the result of one or more conservative amino acid substitutions. It is well known in the art that one or more amino acids in a polypeptide sequence can be substituted with at least one other amino acid having a similar charge and polarity such that the substitution/s result in a silent change in the modified polypeptide that does not alter its function relative to the function of the non-modified sequence. The invention refers to any polypeptide sequence differing in one or more amino acids, either as a result of conserved or non-conserved substitutions, and/or either as a result of sequence insertions or deletions, relative to the sequence given by SEQ ID NO: 1, SEQ ID NO: 2, SEQ ID NO: 3, SEQ ID NO: 4 or SEQ ID NO: 5, as long as said further provided polypeptide sequence has the same or similar or equivalent glycomodule motif as SEQ ID NO: 1, SEQ ID NO: 2, SEQ ID NO: 3, SEQ ID NO: 4 or SEQ ID NO: 5.

By “codon degeneracy” it is meant divergence in the genetic code enabling variation of the nucleotide sequence without affecting the amino acid sequence of an encoded polypeptide. A person skilled in the art is well aware of the codon-bias exhibited by a specific host cell in using nucleotide codons to specify a given amino acid residue. Thus, for ectopic expression of a gene in a host cell, it is desirable to design or synthesize the gene in a way such that its frequency of codon usage approaches the frequency of codon usage of the host cell as described in a codon usage table.

The terms “identity”, “identical” or “percent identity” in the context of two or more amino acid, or nucleotide sequences, refer to two or more sequences or subsequences that are the same or have a specified percentage of amino acid or nucleotide residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences.

The percentage of sequence identity may be determined by comparing two optimally aligned sequences over a comparison window. The aligned sequences may be polynucleotide sequences or polypeptide sequences. For optimal alignment of the two sequences, the portion of the polynucleotide or amino acid sequence in the comparison window may comprise insertions or deletions (i.e., gaps) as compared to the reference sequence (that does not comprise insertions or deletions). The percentage of sequence identity is calculated by determining the number of positions at which the identical nucleotide residues, or the identical amino acid residues, occurs in both compared sequences to yield the number of matched positions, then dividing the number of matched positions by the total number of positions in the window of comparison and multiplying the result by 100 to yield the percentage of sequence identity. Sequence identity between two polypeptide sequences or two polynucleotide sequences can be determined, for example, by using the Gap program in the WISCONSIN PACKAGE version 10.0-UNIX from Genetics Computer Group, Inc. based on the method of Needleman and Wunsch (J. Mol. Biol. 48:443-453, 1970) using the set of default parameters for pairwise comparison (for amino acid sequence comparison: Gap Creation Penalty=8, Gap Extension Penalty=2; for nucleotide sequence comparison: Gap Creation Penalty=50; Gap Extension Penalty=3), or using the TBLASTN program in the BLAST 2.2.1 software suite (Altschul et al., Nucleic Acids Res. 25:3389-3402), using BLOSUM62 matrix (Henikoff and Henikoff, Proc. Natl. Acad. Sci. U.S.A. 89:10915-10919, 1992) and the set of default parameters for pair-wise comparison (gap creation cost=11, gap extension cost=1).

The percentage of sequence identity between polypeptides and their corresponding functions may be determined, for example, using a variety of homology based search algorithms that are available to compare a query sequence, to a protein database, including for example, BLAST, FASTA, and Smith-Waterman. BLASTX and BLASTP algorithms may be used to provide protein function information. A number of values are examined in order to assess the confidence of the function assignment. Useful measurements include “E-value” (also shown as “hit_p”), “percent identity”, “percent query coverage”, and “percent hit coverage”. In BLAST, the E-value, or the expectation value, represents the number of different alignments with scores equivalent to or better than the raw alignment score, S, that are expected to occur in a database search by chance. Hence, the lower the E value, the more significant the match. Since database size is an element in E-value calculations, the E-values obtained by doing a BLAST search against public databases, such as GenBank, have generally increased over time for any given query/entry match. Thus, in setting criteria for confidence of polypeptide function prediction, a “high” BLASTX match is considered as having an E-value for the top BLASTX hit of less than 1E-30; a medium BLASTX is considered as having an E-value of 1E-30 to 1E-8; and a low BLASTX is considered as having an E-value of greater than 1E-8. Percent identity refers to the percentage of identically matched amino acid residues that exist along the length of that portion of the sequences which is aligned by the BLAST algorithm. In setting criteria for confidence of polypeptide function prediction, a “high” BLAST match is considered as having percent identity for the top BLAST hit of at least 70%; a medium percent identity value is considered from 35% to 70%; and a low percent identity is considered of less than 35%. Of particular interest in protein function assignment is the use of combinations of E-values, percent identity, query coverage and hit coverage. Query coverage refers to the percent of the query sequence that is represented in the BLAST alignment, whereas hit coverage refers to the percent of the database entry that is represented in the BLAST alignment. For the purpose of defining the polypeptides functionally covered by the present invention, the function of a polypeptide is deduced from the function of a protein homolog, such as SEQ ID NO: 1, SEQ ID NO: 2, SEQ ID NO: 3, SEQ ID NO: 4 or SEQ ID NO: 5, wherein a polypeptide of the invention is one that either (1) results in hit_p<1e-30 or % identity>35% AND query_coverage>50% AND hit_coverage>50%, or (2) results in hit_p<le-8 AND query_coverage>70% AND hit_coverage>70%. In a preferred embodiment, the sequence identity is determined throughout the whole length of the polypeptide of SEQ ID NO: 1, SEQ ID NO: 2, SEQ ID NO: 3, SEQ ID NO: 4 or SEQ ID NO: 5 or throughout the whole length of the variant or of both.

Functionally equivalent variants of SEQ ID NO: 1 also include sequences with a sequence identity of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% with the sequence SEQ ID NO: 1.

Functionally equivalent variants of SEQ ID NO: 2 also include sequences with a sequence identity of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% with the sequence SEQ ID NO:2.

Functionally equivalent variants of SEQ ID NO: 3 also include sequences with a sequence identity of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% with the sequence SEQ ID NO:2.

Functionally equivalent variants of SEQ ID NO: 4 also include sequences with a sequence identity of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% with the sequence SEQ ID NO:2.

Functionally equivalent variants of SEQ ID NO: 5 also include sequences with a sequence identity of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% with the sequence SEQ ID NO:2.

In a preferred embodiment, the functionally equivalent variant of SEQ ID NO: 1, 2, 3, 4, or 5 has a sequence identity of at least 50% with the corresponding sequence SEQ ID NO, 1, 2, 3, 4 or 5 and the sequence identity is determined throughout the whole length of the sequence SEQ ID NO: 1, 2, 3, 4 or 5.

In another preferred embodiment, the functionally equivalent variant of SEQ ID NO 1, 2, 3, 4 or 5 is a polypeptide sequence having a Methionine residue at the beginning of SEQ ID NO. 1, 2, 3, 4 or 5.

### An Expression Cassette Comprising a Glycomodule Motif

In a second aspect the invention relates to an expression cassette comprising at least one nucleotide sequence encoding a glycomodule motif according to the first aspect.

“An expression cassette” as disclosed herein refers to a component of a vector DNA comprising one or more genes and the sequences controlling their expression. Non-limiting basic components of an expression cassette include promoter elements, the gene(s) of interest, and an appropriate mRNA stabilizing polyadenylation signal. Other frequently employed cis-acting elements include internal ribosome entry site (IRES) sequences to allow expression of two or more genes without the need for an additional promoter, introns and post-transcriptional regulatory elements to improve transgene expression.

In a preferred embodiment, the expression cassette of the invention further comprises
(i) a nucleotide sequence encoding a secretory signal peptide,
(ii) a nucleotide sequence encoding a selectable marker,
(iii) a nucleotide sequence encoding a tag,
(iv) a regulatory nucleotide sequence,
(v) a nucleotide sequence encoding a protease recognition site,
(vi) a nucleotide sequence encoding a protein of interest or
(vii) any combination thereof.

In a more preferred embodiment, the expression cassette of the invention further comprises two sequences selected from the group consisting of a nucleotide sequence encoding a secretory signal peptide, a nucleotide sequence encoding a selectable marker, a nucleotide sequence encoding a tag, a regulatory nucleotide sequence, a nucleotide sequence encoding a protease recognition site and a nucleotide sequence encoding a protein of interest. Particularly the expression cassette of the invention comprises a nucleotide sequence encoding a secretory signal peptide and a nucleotide sequence encoding a selectable marker, a nucleotide sequence encoding a secretory signal peptide and a nucleotide sequence encoding a tag, a nucleotide sequence encoding a secretory signal peptide and a regulatory nucleotide sequence, a nucleotide sequence encoding a secretory signal peptide and a nucleotide sequence encoding a protease recognition site, a nucleotide sequence encoding a secretory signal peptide and a nucleotide sequence encoding a protein of interest, a nucleotide sequence encoding a selectable marker and a nucleotide sequence encoding a tag, a nucleotide sequence encoding a selectable marker and a regulatory nucleotide sequence, a nucleotide sequence encoding a selectable marker and a nucleotide sequence encoding a protease recognition site, a nucleotide sequence encoding a selectable marker and a nucleotide sequence encoding a protein of interest, a nucleotide sequence encoding a tag and a regulatory nucleotide sequence, a nucleotide sequence encoding a tag and a nucleotide sequence encoding a protease recognition site, a nucleotide sequence encoding a tag and a nucleotide sequence encoding a protein of interest, a regulatory nucleotide sequence and a nucleotide sequence encoding a protease recognition site, a regulatory nucleotide sequence and a nucleotide sequence encoding a protein of interest, or a nucleotide sequence encoding a protease recognition site and a nucleotide sequence encoding a protein of interest.

In another preferred embodiment, the expression cassette of the invention further comprises three sequences selected from the group consisting of a nucleotide sequence encoding a secretory signal peptide, a nucleotide sequence encoding a selectable marker, a nucleotide sequence encoding a tag, a regulatory nucleotide sequence, a nucleotide sequence encoding a protease recognition site and a nucleotide sequence encoding a protein of interest. Particularly, the expression cassette of the invention further comprises a nucleotide sequence encoding a secretory signal peptide, a nucleotide sequence encoding a selectable marker and a nucleotide sequence encoding a tag; a nucleotide sequence encoding a secretory signal peptide, a nucleotide sequence encoding a selectable marker and a regulatory nucleotide sequence; a nucleotide sequence encoding a secretory signal peptide, a nucleotide sequence encoding a selectable marker and a nucleotide sequence encoding a protease recognition site; a nucleotide sequence encoding a secretory signal peptide, a nucleotide sequence encoding a selectable marker and a nucleotide sequence encoding a protein of interest; a nucleotide sequence encoding a secretory signal peptide, a nucleotide sequence encoding a tag and a regulatory nucleotide sequence; a nucleotide sequence encoding a secretory signal peptide, a nucleotide sequence encoding a tag and a nucleotide sequence encoding a protease recognition site; a nucleotide sequence encoding a secretory signal peptide, a nucleotide sequence encoding a tag and a nucleotide sequence encoding a protein of interest; a nucleotide sequence encoding a secretory signal peptide, a regulatory nucleotide sequence and a nucleotide sequence encoding a protease recognition site; a nucleotide sequence encoding a secretory signal peptide, a regulatory nucleotide sequence and a nucleotide sequence encoding a protein of interest; a nucleotide sequence encoding a secretory signal peptide, a nucleotide sequence encoding a protease recognition site and a nucleotide sequence encoding a protein of interest; a nucleotide sequence encoding a selectable marker, a nucleotide sequence encoding a tag and a regulatory nucleotide sequence; a nucleotide sequence encoding a selectable marker, a nucleotide sequence encoding a tag and a nucleotide sequence encoding a protease recognition site; a nucleotide sequence encoding a selectable marker, a nucleotide sequence encoding a tag and a nucleotide sequence encoding a protein of interest; a nucleotide sequence encoding a selectable marker, a regulatory nucleotide sequence and a nucleotide sequence encoding a protease recognition site; a nucleotide sequence encoding a selectable marker, a regulatory nucleotide sequence and a nucleotide sequence encoding a protein of interest; a nucleotide sequence encoding a selectable marker, a nucleotide sequence encoding a protease recognition site and a nucleotide sequence encoding a protein of interest; a nucleotide sequence encoding a tag, a regulatory nucleotide sequence and a nucleotide sequence encoding a protease recognition site; a nucleotide sequence encoding a tag, a regulatory nucleotide sequence and a nucleotide sequence encoding a protein of interest; a nucleotide sequence encoding a tag, a nucleotide sequence encoding a protease recognition site and a nucleotide sequence encoding a protein of interest; or a regulatory nucleotide sequence, a nucleotide sequence encoding a protease recognition site and a nucleotide sequence encoding a protein of interest.

In another preferred embodiment, the expression cassette of the invention further comprises four sequences selected from the group consisting of a nucleotide sequence encoding a secretory signal peptide, a nucleotide sequence encoding a selectable marker, a nucleotide sequence encoding a tag, a regulatory nucleotide sequence, a nucleotide sequence encoding a protease recognition site and a nucleotide sequence encoding a protein of interest. Particularly, the expression cassette of the invention further comprises a nucleotide sequence encoding a secretory signal peptide, a nucleotide sequence encoding a selectable marker, a nucleotide sequence encoding a tag and a regulatory nucleotide sequence; a nucleotide sequence encoding a secretory signal peptide, a nucleotide sequence encoding a selectable marker, a nucleotide sequence encoding a tag and a nucleotide sequence encoding a protease recognition site; a nucleotide sequence encoding a secretory signal peptide, a nucleotide sequence encoding a selectable marker, a nucleotide sequence encoding a tag and a nucleotide sequence encoding a protein of interest; a nucleotide sequence encoding a secretory signal peptide, a nucleotide sequence encoding a selectable marker, a regulatory nucleotide sequence and a nucleotide sequence encoding a protease recognition site; a nucleotide sequence encoding a secretory signal peptide, a nucleotide sequence encoding a selectable marker, a regulatory nucleotide sequence and a nucleotide sequence encoding a protein of interest; a nucleotide sequence encoding a secretory signal peptide, a nucleotide sequence encoding a selectable marker, a nucleotide sequence encoding a protease recognition site and a nucleotide sequence encoding a protein of interest; a nucleotide sequence encoding a secretory signal peptide, a nucleotide sequence encoding a tag, a regulatory nucleotide sequence and a nucleotide sequence encoding a protease recognition site; a nucleotide sequence encoding a secretory signal peptide, a nucleotide sequence encoding a tag, a regulatory nucleotide sequence and a nucleotide sequence encoding a protein of interest; a nucleotide sequence encoding a secretory signal peptide, a nucleotide sequence encoding a tag, a nucleotide sequence encoding a protease recognition site and a nucleotide sequence encoding a protein of interest; a nucleotide sequence encoding a secretory signal peptide, a regulatory nucleotide sequence a nucleotide sequence encoding a protease recognition site and a nucleotide sequence encoding a protein of interest; a nucleotide sequence encoding a selectable marker, a nucleotide sequence encoding a tag, a regulatory nucleotide sequence and a nucleotide sequence encoding a protease recognition site; a nucleotide sequence encoding a selectable marker, a nucleotide sequence encoding a tag, a regulatory nucleotide sequence and a nucleotide sequence encoding a protein of interest; a nucleotide sequence encoding a selectable marker, a nucleotide sequence encoding a tag, a nucleotide sequence encoding a protease recognition site and a nucleotide sequence encoding a protein of interest; a nucleotide sequence encoding a selectable marker, a regulatory nucleotide sequence, a nucleotide sequence encoding a protease recognition site and a nucleotide sequence encoding a protein of interest; or a nucleotide sequence encoding a tag, a regulatory nucleotide sequence, a nucleotide sequence encoding a protease recognition site and a nucleotide sequence encoding a protein of interest.

In a more preferred embodiment, the expression cassette of the invention further comprises five sequences selected from the group consisting of a nucleotide sequence encoding a secretory signal peptide, a nucleotide sequence encoding a selectable marker, a nucleotide sequence encoding a tag, a regulatory nucleotide sequence, a nucleotide sequence encoding a protease recognition site and a nucleotide sequence encoding a protein of interest. Particularly the expression cassette of the invention comprises a nucleotide sequence encoding a secretory signal peptide, a nucleotide sequence encoding a selectable marker, a nucleotide sequence encoding a tag, a regulatory nucleotide sequence and a nucleotide sequence encoding a protease recognition site; a nucleotide sequence encoding a secretory signal peptide, a nucleotide sequence encoding a selectable marker, a nucleotide sequence encoding a tag, a regulatory nucleotide sequence and a nucleotide sequence encoding a protein of interest; a nucleotide sequence encoding a secretory signal peptide, a nucleotide sequence encoding a selectable marker, a nucleotide sequence encoding a tag, a nucleotide sequence encoding a protease recognition site and a nucleotide sequence encoding a protein of interest; a nucleotide sequence encoding a secretory signal peptide, a nucleotide sequence encoding a selectable marker, a regulatory nucleotide sequence, a nucleotide sequence encoding a protease recognition site and a nucleotide sequence encoding a protein of interest; a nucleotide sequence encoding a secretory signal peptide, a nucleotide sequence encoding a tag, a regulatory nucleotide sequence, a nucleotide sequence encoding a protease recognition site and a nucleotide sequence encoding a protein of interest; or a nucleotide sequence encoding a selectable marker, a nucleotide sequence encoding a tag, a regulatory nucleotide sequence, a nucleotide sequence encoding a protease recognition site and a nucleotide sequence encoding a protein of interest.

In a more preferred embodiment, the expression cassette of the invention further comprises a nucleotide sequence encoding a secretory signal peptide, a nucleotide sequence encoding a selectable marker, a nucleotide sequence encoding a tag, a regulatory nucleotide sequence, a nucleotide sequence encoding a protease recognition site and a nucleotide sequence encoding a protein of interest.

Fusion of signal peptide to the reporter protein results in secretion of the fusion protein to media, which is the preferred strategy since it permits easy and efficient purification from the extracellular medium. In addition, the secretory production of recombinant proteins has the advantage that proteolytic degradation may be avoided and that there is a better chance of correct protein folding.

In a preferred embodiment the expression cassette besides the glycomodule motif of the invention further comprises a nucleotide sequence encoding a secretory signal peptide. As it is used herein, the term “signal peptide” or “secretory signal peptide” refers to a peptide of a relatively short length, generally between 5 and 30 amino acid residues, directing proteins synthesized in the cell towards the secretory pathway. The signal peptide usually contains a series of hydrophobic amino acids adopting a secondary alpha helix structure. Additionally, many peptides include a series of positively-charged amino acids that can contribute to the protein adopting the suitable topology for its translocation. The signal peptide tends to have at its carboxyl end a motif for recognition by a peptidase, which is capable of hydrolyzing the signal peptide giving rise to a free signal peptide and a mature protein. The nucleotide sequence encoding a signal peptide is operatively linked to the nucleotide sequence encoding the protein of interest. The signal peptide can be cleaved from the protein of interest once it has reached the appropriate location. Any secretory signal peptide may be used in the present invention, such as a way of illustrative non limitative example signal peptide from Chlamydomonas reinhardtii carbonic anhydrase (CAH1) (SEQ ID NO: 11) having a nucleotide sequence shown in SEQ ID NO: 10, signal peptide from Chlamydomonas reinhardtii periplasmic arylsulfatase (ARS1) (SEQ ID NO: 13), having a nucleotide sequence shown in SEQ ID NO: 12 or the signal peptide from Chlamydomonas reinhardtii Gametolysin M11 (SEQ ID NO: 15) having a nucleotide sequence shown in SEQ ID NO:14.

The presence of a selectable marker allows a fast and easy monitorization of highly expressing transformants. Thus, in another preferred embodiment, the expression cassette besides the polynucleotide sequence encoding the glycomodule motif of the invention further comprises a nucleotide sequence encoding a selectable marker. As used herein, a “selectable marker” or reporter gene is a gene, to a protein that typically is not present in the recipient organism and typically encodes for proteins resulting in some phenotypic change or enzymatic property which may allow for the selection of transformed cells, the expression of which creates a detectable phenotype and which facilitates detection of host cells that contain an expression cassette having the selectable marker or reporter gene. Non-limiting examples of selectable markers include drug resistance genes and nutritional markers. For example, the selectable marker can be a gene that confers resistance to an antibiotic selected from the group consisting of: ampicillin, kanamycin, erythromycin, chloramphenicol, gentamycin, kasugamycin, rifampicin, spectinomycin, D-Cycloserine, nalidixic acid, streptomycin, hygromycin or tetracycline, or to herbicides such as acetoliasa synthase gene (ALS) which confers resistance to the herbicide silfonilurea, or the BAR gene conferring resistence to the herbicide phosphinothricin (PPT). Other non-limiting examples of selection markers include adenosine deaminase, aminoglycoside phosphotransferase, dihydrofolate reductase, hygromycin-B-phosphotransferase, thymidine kinase, and xanthine-guanine phosphoribosyltransferase. A single expression cassette can comprise one or more selectable markers. In a preferred embodiment, the expression cassette of the invention comprises as a selectable maker luciferase (luc) genes from Gaussia princess (SEQ ID NO: 35) In another preferred embodiment, the expression cassette of the invention comprises as a selectable marker a nucleotide sequence of shBle gene (SEQ ID NO: 16) or a nucleotide sequence of shBle gene containing the sequence of RBCS2 intron (SEQ ID NO: 36) that codes for bleomycin resistance and can be selected for using bleomycin, a neo gene that codes for kanamycin resistance and can be selected for using kanamycin, G418, etc. Non-limiting-examples of selectable marker genes also include nucleotide sequences encoding a reporter protein. Examples of such genes are provided in K. Wising et al. Ann. Rev. Genetics, 22, 421 (1988). Non-limiting examples of reporter genes include the beta-glucuronidase (GUS) of the uidA locus of E. coli, the chloramphenicol acetyl transferase gene from Tn9 of E. coli, the green fluorescent protein from the bioluminescent jellyfish Aequorea victoria, and the luciferase (luc) genes from Gaussia princess. An assay for detecting reporter gene expression may then be performed at a suitable time after said gene has been introduced into recipient cells. One preferred assay entails the use of the gene encoding beta-glucuronidase (GUS) of the uidA locus of E. coli as described by Jefferson et al., (Biochem. Soc. Trans. 15, 17-19 (1987) to identify transformed cells, referred to herein as GUS:1. A reporter protein may be used with or without an additional selectable marker.

In another preferred embodiment, the expression cassette besides the polynucleotide sequence encoding the glycomodule motif of the invention further comprises a nucleotide sequence encoding a tag. As it is used herein, the term “tag” means a polypeptide useful for making the detection, isolation and/or purification of a protein easier. Generally, said labeling sequence is located in a part of the protein of interest that does not adversely affect the functionality thereof. Virtually any polypeptide which can be used for detecting, isolating and/or purifying a protein can be present in the protein of interest. By way of non-limiting illustration, said polypeptide useful for detecting, isolating and/or purifying a protein, such as a protein of interest, can be, for example, an arginine tag (Arg-tag), a histidine tag (His-tag), FLAG-tag, Strep-tag, an epitope susceptible to being recognized by an antibody, such as c-myc-tag, SBP-tag, S-tag, calmodulin-binding peptide, cellulose-binding domain, chitin-binding domain, glutathione S-transferase-tag, maltose-binding protein, NusA, TrxA, DsbA, Avi-tag, etc. (Terpe K., 2003, Appl. Microbiol. Biotechnol. 60: 523-525), β-galactosidase, VSV-glycoprotein, etc.). In a more preferred embodiment of the expression cassette of the invention, the nucleotide sequence encoding a tag is selected from the group consisting of a nucleotide sequence encoding a hexahistidine tag and/or a 3×HA tag. As disclose herein a “hexahistidine tag”, “6×His-tag” or “polyhistidine-tag” is an amino acid motif in proteins that consists of at least six histidine (His) residues, often at the N- or C-terminus of the protein. As disclosed herein a “3×HA tag” or “3× Hemagglutinin tag”, is an amino acid sequence derived from the Human influenza hemagglutinin-molecule corresponding to amino acids 98-106. In a preferred embodiment the hexahistidine tag is located between the signal sequence and the selectable marker. In another preferred embodiment the 3×HA tag is located between the selectable marker and the gene of interest. In a more preferred embodiment the expression cassette comprises the hexahistidine tag and the 3×HA tag. In a still more preferred embodiment the hexahistidine tag is located between the signal sequence and the selectable marker and the 3×HA tag is located between the selectable marker and the gene of interest. In a still more preferred embodiment a protease recognition sequence is located between the 3×HA tag and the gene of interest.

In another preferred embodiment, the expression cassette of the invention besides the polynucleotide sequence encoding the glycomodule motif further comprises a regulatory nucleotide sequence. As it is used herein, the term “regulatory nucleotide sequence” refers to nucleic acid regions located upstream (5′ non-coding sequences), within, or downstream (3′ non-coding sequences) of a coding region, and which influence the transcription, RNA processing or stability, or translation of the associated coding region. Regulatory regions may include promoters, translation leader sequences, RNA processing site, effector binding site and stem-loop structure. The boundaries of the coding region are determined by a start codon at the 5′ (amino) terminus and a translation stop codon at the 3′ (carboxyl) terminus. A coding region can include, but is not limited to, prokaryotic regions, cDNA from mRNA, genomic DNA molecules, synthetic DNA molecules, or RNA molecules. If the coding region is intended for expression in a eukaryotic cell, a polyadenylation signal and transcription termination sequence will usually be located 3′ to the coding region.

In a preferred embodiment of the expression cassette of the invention the regulatory nucleotide sequence is selected from the group consisting of a promoter sequence, a 5′UTR, a 3′UTR, a flanking region, an intron and any combination thereof. In a more preferred embodiment, said regulatory nucleotide sequence is a Chlamydomonas regulatory sequence.

As it is used herein, the term “promoter” refers to a nucleic acid sequence which is structurally characterized by the presence of a binding site for the DNA-dependent RNA polymerase, transcription start sites and any other DNA sequence including, but without being limited to, transcription factor binding sites, repressor and activator protein binding sites and any other nucleotide sequence known in the state of the art capable of directly or indirectly regulating transcription from a promoter. Promoter refers to a DNA fragment capable of controlling the expression of a coding sequence or functional RNA. In general, a coding region is located 3′ to a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity. A promoter is generally bounded at its 3′ terminus by the transcription initiation site and extends upstream (5′ direction) to include the minimum number of bases or elements necessary to initiate transcription at levels detectable above background. Within the promoter will be found a transcription initiation site (conveniently defined for example, by mapping with nuclease S1), as well as protein binding domains (consensus sequences) responsible for the binding of RNA polymerase. In a more preferred embodiment, a promoter of the expression cassette of the invention is the selected RPL23 promoter (SEQ ID NO: 17), the ferredoxin 1 FDX promoter (SEQ ID NO: 18) or the HSP70-RCBS2 chimeric promoter, also known as AR (SEQ ID NO: 19).

As it is used herein, the term “5′-UTR”, refers to the sequence at the 5′ end of the expression cassette which is not translated and which contains the region necessary for replication, i.e., the sequence which is recognized by the polymerase during synthesis of the RNA molecule from the RNA template. In a preferred embodiment, the 5′ untranslated sequence is selected from the group consisting of RPL23 5′UTR, SEQ ID NO: 29), the ferredoxin 1 FDX5′UTR (SEQ ID NO: 30) or the RCBS2 5′ UTR (SEQ ID NO: 31).

In another preferred embodiment, the regulatory nucleotide sequence at 5′ is selected from the group consisting of RPL23 promoter+5′ RPL23 5′UTR (SEQ ID NO: 37), FDX promoter+5′UTR (SEQ ID NO: 39) and HSP70-RCBS2 chimeric promoter+RCBS2 5′ UTR (SEQ ID NO: 41).

As it is used herein, the term “3′-UTR”, refers to an untranslated region which appears after the end codon. The 3′ untranslated region typically contains a polyadenine tag which allows increasing RNA stability, and therefore the amount of products resulting from the translation of said RNA. The poly(A) tag can be of any size provided that it is sufficient to increase stability in the cytoplasm of the molecule of the vector of the invention. In a preferred embodiment, the 3′ untranslated sequence is selected from the group consisting of 3′UTR of RPL23 (SEQ ID NO: 20), 3′UTR of the RCBS2 gene (SEQ ID NO: 21) and the 3′UTR of the FDX gene promoter (SEQ ID NO: 22).

In another preferred embodiment, the expression cassette of the invention comprises the 3′ untranslated sequence, a terminator and additional flanking regions. In a preferred embodiment, the terminator and flanking regions are selected from the group consisting of SEQ ID NO: 23, 32 and 33.

As it is used herein, the term “flanking region” refers to a DNA sequence extending on either side of a specific sequence. Flanking regions may be adjacent to the promoter, 5′UTR, or 3′UTR sequences, used in the present invention.

Thus, in a preferred embodiment the sequence comprising a 3′UTR and terminator and flanking regions that can be used in the present invention is selected from the group consisting of SEQ ID NO: 38, 40 and 42.

In a preferred embodiment, the regulatory sequence of the expression cassette of the invention comprises a sequence selected from the group consisting of HSP70A-RCBS2 chimeric promoter (SEQ ID NO: 19), the FDX gene promoter (SEQ ID NO: 18), the RPL23 promoter (SEQ ID NO: 17), PL23 promoter+5′ RPL23 5′UTR (SEQ ID NO: 37), FDX promoter+5′UTR (SEQ ID NO: 39), HSP70-RCBS2 chimeric promoter+RCBS2 5′ UTR (SEQ ID NO: 41), the 3′UTR of the RCBS2 gene (SEQ ID NO: 21), the 3′UTR of the FDX gene (SEQ ID NO:22), the 3′UTR of RPL23 (SEQ ID NO: 20), SEQ ID NO: 38, 40 and 42 and any combination thereof. In another preferred embodiment, the regulatory sequence of the expression cassette of the invention is selected from the group consisting of HSP70A-RCBS2 chimeric promoter (SEQ ID NO: 19), the FDX gene promoter (SEQ ID NO: 18), the RPL23 promoter (SEQ ID NO: 17), PL23 promoter+5′ RPL23 5′UTR (SEQ ID NO: 37), FDX promoter+5′UTR (SEQ ID NO: 39), HSP70-RCBS2 chimeric promoter+RCBS2 5′ UTR (SEQ ID NO: 41), the 3′UTR of the RCBS2 gene (SEQ ID NO: 21), the 3′UTR of the FDX gene (SEQ ID NO:22), the 3′UTR of RPL23 (SEQ ID NO: 20), SEQ ID NO: 38, 40 and 42 and any combination thereof.

As it is used herein, the term “intron” refers to any nucleotide sequence within a gene that is removed by RNA splicing during maturation of the final RNA product. The term intron refers to both the DNA sequence within a gene and the corresponding sequence in RNA transcripts. In a preferred embodiment the intron is inserted into the nucleotide sequence encoding a selectable marker. In another preferred embodiment, the intron sequence is inserted into the promoter sequence. In a preferred embodiment, the intron is from a highly expressed gene. In a more preferred embodiment the intron is a RCBS2 intron having the sequence shown in SEQ ID NO: 24.

In a preferred embodiment, the expression cassette besides the polynucleotide sequence encoding the glycomodule motif further comprises a nucleotide sequence encoding a protease recognition site, wherein said nucleotide sequence encoding a protease recognition site is placed in the same open reading frame as the two nucleotide sequences encoding the proteins that are to be separated as a result of the protease activity. The inclusion of a protease sequence in the expression cassette allows the release of the protein of interest from sequences that may interfere with activity.

As it is used herein, the term “protease recognition site” refers to an amino acid sequence which is susceptible to being cleaved by an enzyme that performs proteolysis, protein catabolism by hydrolysis of peptide bonds, once the protein has been translated. An illustrative space is an amino acid sequence that is cleavable by a protease such as an enterokinase, Arg-C endoprotease, Glu-C endoprotease, Lys-C endoprotease, Factor Xa, SUMO proteases (Tauseef et al., 2005 Protein Expr. Purif. 43:1-9) and the like. In a preferred embodiment the protease recognition sequence is a plant specific protease recognition sequence. In another preferred embodiment the protease recognition sequence is the TEV (Tobacco Etch Virus nuclear-inclusion-α endopeptidase) protease recognition sequence SEQ ID NO: 25.

In a preferred embodiment, the expression cassette besides the polynucleotide sequence encoding the glycomodule motif further comprises a nucleotide sequence encoding a protein of interest, which is located in same open reading frame as the polynucleotide encoding the glycomodule so that the expression of the cassette results in the expression of a fusion protein comprising the glycomodule and the protein of interest. The term “protein of interest” refers to any protein the expression of which in a cell is to be achieved. In a preferred embodiment, the protein of interest is heterologous. Heterologous sequence could be a sequence that is derived from a different gene or from the same host, from a different strain of host cell, or from an organism of a different taxonomic group (e.g., different kingdom, phylum, class, order, family genus, or species, or any subgroup within one of these classifications). The term “heterologous” is also used synonymously herein with the term “exogenous.” In a preferred embodiment, the protein of interest is in the form of a precursor. The term “precursor” refers to a polypeptide which, once processed, can give rise to a protein of interest. In a particular embodiment, the precursor of the protein of interest is a polypeptide comprising a signal sequence or signal peptide. In a preferred embodiment, the protein of interest is the epidermal growth factor (EGF), more preferably from human. EGF as used herein relates to a 6 kDa protein that stimulates cell growth and differentiation that in human corresponds to the sequence with accession number Q6QBS2 in the Uniprot database 28 Feb. 2018. In a preferred embodiment, the nucleotide sequence coding human EGF is the sequence shown in SEQ ID NO: 34.

In another preferred embodiment the expression cassette comprises a nucleotide sequence encoding a linker. As it is used herein, the term “linker” means a suitable peptide that allows for two or more functional domains joined together in a fusion protein. Linkers can be flexible or rigid linkers. It will be understood that the nucleotide sequence encoding the linker will be found in the expression cassette in the same open reading frame as the two nucleotide sequences which encode the functional domains that are connected by the linker. In a preferred embodiment the linker is a flexible linker. “Flexible linker” as it is used herein means that the joined domains require a certain degree of movement or interaction. They are generally composed of small, non-polar (e.g. Gly) or polar (e.g. Ser or Thr) amino acids. The small size of these amino acids provides flexibility, and allows for mobility of the connecting functional domains. The incorporation of Ser or Thr can maintain the stability of the linker in aqueous solutions by forming hydrogen bonds with the water molecules, and therefore reduces the unfavorable interaction between the linker and the protein moieties. The most commonly used flexible linkers have sequences consisting primarily of stretches of Gly and Ser residues (“GS” linker). By adjusting the copy number “n”, the length of this GS linker can be optimized to achieve appropriate separation of the functional domains, or to maintain necessary inter-domain interactions. In a more preferred embodiment the linker is (GGGGS)n SEQ ID NO:26. In a more preferred embodiment, the nucleotide sequence encoding a linker is included between the nucleotide sequence encoding a glycomodule motif and the nucleotide sequence encoding a protease recognition sequence.

The expression cassette of the invention may contain the additional elements selected from the group consisting of: a nucleotide sequence encoding a signal peptide, a nucleotide sequence encoding a selectable marker, a nucleotide sequence encoding a tag, a regulatory nucleotide sequence, a nucleotide sequence encoding a protease recognition site and a nucleotide sequence encoding a protein of interest an any combinations thereof located at any position in the cassette.

In a preferred embodiment, the expression cassette comprises from 5′ to 3′ in the same open reading frame a nucleotide sequence encoding a glycomodule motif and a nucleotide sequence encoding a protein of interest. As it is used herein, the term “open reading frame” or “ORF” means a length of nucleic acid, either DNA, cDNA or RNA, that comprises a translation start signal or initiation codon, such as an ATG or AUG, and a termination codon and can be potentially translated into a polypeptide sequence. Said DNA sequence does not contain any internal end codon and can generally be translated into a peptide.

In a preferred embodiment, the expression cassette comprises from 5′ to 3′ in the same open reading frame a nucleotide sequence encoding a selectable marker, a nucleotide sequence encoding a glycomodule motif and a nucleotide sequence encoding a protein of interest. In another preferred embodiment, the expression cassette comprises from 5′ to 3′ in the same open reading frame a nucleotide sequence encoding a glycomodule motif and a nucleotide sequence encoding a protein of interest further comprising a nucleotide sequence encoding a selectable marker in the same open reading frame as the nucleotide sequence encoding a glycomodule motif and the nucleotide sequence encoding a protein of interest.

In a preferred embodiment, the expression cassette comprises from 5′ to 3′ a nucleotide sequence encoding a signal peptide, a nucleotide sequence encoding a first tag, a nucleotide sequence encoding a selectable marker, a nucleotide sequence encoding a second tag, a nucleotide sequence encoding a protein of interest and a nucleotide sequence encoding a glycomodule motif. In a more preferred embodiment, all elements are in the same open reading frame. In a still more preferred embodiment, all elements are under the operation control of a regulatory nucleotide sequence. In a preferred embodiment, the first tag is different from the second tag.

In another preferred embodiment, the nucleotide sequence encoding a protease recognition site is located between the nucleotide sequence encoding a second tag and the nucleotide sequence encoding the protein of interest. In another preferred embodiment, the nucleotide sequence encoding a protease recognition site is located between the nucleotide sequence encoding a second tag and before the nucleotide sequence encoding a glycomodule motif.

In a particular embodiment, the expression cassette of the invention comprises at least one additional nucleotide sequence encoding a second glycomodule motif. In a preferred embodiment, the additional nucleotide sequence encoding a second glycomodule motif is different from the nucleotide sequence of the first glycomodule of the expression cassette of the invention.

The use of more than one glycomodule motif may be beneficial for protein stability and having different glycomodule sequences in the same cassette is advantageous because it can avoid DNA recombination during vector cloning, preparation or cell transformation.

In a preferred embodiment, the additional nucleotide sequence encoding a second glycomodule motif is selected from the group consisting of SEQ ID NO: 1, SEQ ID NO: 2, SEQ ID NO 3, SEQ ID NO 4, SEQ ID NO 5, any functionally equivalent variant thereof and (SP)n. (SP)n as disclosed herein refers to a nucleic acid construct that codes for n-repeating units of Serine-Proline, as disclosed in U.S. Pat. No. 9,006,410B2. In a preferred embodiment the second glycomodule motif is selected from the group consisting of: SEQ ID NO 1, SEQ ID NO 2, SEQ ID NO 3, SEQ ID NO 4, SEQ ID NO 5 and any functionally equivalent variant thereof. In another preferred embodiment the second glycomodule motif is (SP)n. In a more preferred embodiment the n-repeting units is between 5 and 30. In a still more preferred embodiment the n-repeting units is 10 or 20 ((SP)₁₀ (SEQ ID NO: 27 or (SP)₂₀ SEQ ID NO: 28). In a more preferred embodiment, the expression cassette comprises the (SP)₂₀ SEQ ID NO: 28 and SEQ ID NO:1. In another more preferred embodiment, the expression cassette comprises the (SP)₂₀ SEQ ID NO: 28 and SEQ ID NO:2. In another more preferred embodiment, the expression cassette comprises the (SP)₂₀ SEQ ID NO: 28 and SEQ ID NO:3. In another more preferred embodiment, the expression cassette comprises the (SP)₂₀ SEQ ID NO: 28 and SEQ ID NO:4. In another more preferred embodiment, the expression cassette comprises the (SP)₂₀ SEQ ID NO: 28 and SEQ ID NO:5.

The invention also relates to an expression cassette comprising from 5′ to 3′ in the same open reading frame, a nucleotide sequence encoding a selectable marker, a nucleotide sequence encoding a glycomodule motif and a nucleotide sequence encoding a protein of interest. In a particular embodiment, the nucleotide sequence encoding a glycomodule motif is a nucleotide sequence encoding (SP)ₙ, particularly (SP)₁₀ or (SP)₂₀.

All the terms and embodiments previously described are equally applicable to this aspect of the invention.

### Vector Comprising a Glycomodule Motif

In another aspect the invention relates to a vector comprising a nucleotide sequence encoding a glycomodule motif of the invention or an expression cassette according to the invention.

As it is used herein, the term “vector” or “expression vector” refers to a replicative DNA construct used for expressing the glycomodule motif or the expression cassette of the invention in a cell, preferably a eukaryotic cell. The choice of expression vector will depend upon the choice of host. A wide variety of expression host/vector combinations can be employed. Useful expression vectors for eukaryotic hosts, include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR 1, pBR322, pMB9 and their derivatives, wider host range plasmids, such as M13 and filamentous single-stranded DNA phages. In a preferred embodiment, the vector is suitable for expression in microalga. Preferred vectors for this invention are vectors developed for algae such as the vectors commonly known by the skilled person such as pChlamy_4 vector (Invitrogen), or vectors available through Chlamydomonas center.

These vectors may contain an additional independent cassette to express a selectable marker different from the selectable marker of the expression cassette comprising the sequence coding for the protein of interest, which will be used to initially selecting clones that have incorporated the exogenous DNA during the transformation protocol. In a more preferred embodiment, the selectable marker is a resistance gene, more preferably a gene that confers resistance to an antibiotic, more preferably resistance to hygromycin. In a more preferred embodiment, the additional cassette has the sequence shown in SEQ ID NO: 43, comprising the beta tubulin promoter, the APH7 sequence containing an intron of RBCS2 and 3′UTR RBCS2.

The expression vector preferably contains an origin of replication in prokaryotes, necessary for vector propagation in bacteria. Additionally, the expression vector can also contain a selection gene for bacteria, for example, a gene encoding a protein conferring resistance to an antibiotic, for example, ampicillin, kanamycin, chloramphenicol, etc.

The expression vector may contain an origin of replication in microalga. The expression vector can also contain one or more multiple cloning sites. A multiple cloning site is a polynucleotide sequence comprising one or more unique restriction sites. Non-limiting examples of the restriction sites include EcoRI, SacI, KpnI, SmaI, XmaI, BamHI, XbaI, HincII, PstI, SphI, HindIII, AvaI, or any combination thereof.

All the terms and embodiments previously described are equally applicable to this aspect of the invention.

### Host Cell

In another aspect the invention relates to a host cell comprising a vector as described previously.

The term “host cell” is used such that it refers not only to the particular subject cell, but to the progeny or potential progeny of such a cell. Because certain modifications may occur in succeeding generations due to either mutation or environmental influences, such progeny may not, in fact, be identical to the parent cell, but are still included within the scope of the term as used herein. A host cell can be any prokaryotic (e.g., E. coli) or eukaryotic cell (e.g., yeast or plant cells). In a preferred embodiment the host cell is a microalga. Microalga as used herein relates to large and diverse group of simple, typically autotrophic organisms, ranging from unicellular to multicellular forms, microscopic algae, typically found in freshwater and marine systems. Examples of suitable microalgae for obtaining a recombinant protein of the invention, include microalgae from the phylums Cyanophyta, Chlorophyta, Rhodophyta, Heterokontophyta, and Haptophyta. The algae from the phylum Cyanophyta can be Spirulina (Arthrospira), Aphanizomenon flos-aquae, Anabaena cylindrica or Lyngbya majuscule. The algae from the phylum Chlorophyta can be Chlorella, Scenedesmus, Dunaliella, Tetraselmis, Haematococcus, Ulva, Codium, Botryococcus or Caulerpa spp. the algae from the phylum Rhodophyta can be Porphyridium cruentum, Gracilaria sp., Grateloupia sp, Palmaria sp. Corallina sp., Chondrus crispus, Porphyra sp. or Rhodosorus sp. The algae from the phylum Heterokontophyta can be Nannochlorropsis oculata, Odontella aurita, Phaeodactylum tricornutum. Fucus sp. Sargassum sp. Padina sp., Undaria pinnatifida, or Laminaria sp. The algae from the phylum Haptophyta can be Isochrysis sp. Tisochrysis sp. or Pavlova sp. The algae can be Chrypthecodinium cohnii, Schizochytrium, Ulkenia or Euglena gracilis. The algae can be a green microalga such as Chlorella, Scenedesmus, Dunialiella, Haematococcus and Bracteacoccus; haptophyte microalgae such as Isochrysis; and heterokontophyta microalgae such as Phaeodactylum, Ochromonas and Odontella.

In a particular embodiment, the microalga is a green alga. Suitable examples of green alga are Chlorella or Haematyococcus, Botryococcus or Chlamydomonas. In another preferred embodiment, the microalga is from genus Chlamydomonas.

Chlamydomonas, as used herein relates to a genus of green algae consisting of about 325 species all unicellular flagellates, found in stagnant water and on damp soil, in freshwater, seawater, and even in snow as “snow algae”. In a more preferred embodiment, the microalga is Chlamydomonas reinhardtii. In another preferred embodiment, the microalga is Botryococcus braunii.

Chlamydomonas reinhardtii, as used herein is a single-cell green alga about 10 micrometres in diameter that swims with two flagella. It has a cell wall made of hydroxyproline-rich glycoproteins, a large cup-shaped chloroplast, a large pyrenoid, and an “eyespot” that senses light.

All the terms and embodiments previously described are equally applicable to this aspect of the invention.

### A Method for Expressing a Protein of Interest

In another aspect the invention relates to a method for expressing a protein of interest which comprises growing a microalga cell comprising a vector according to the invention, wherein the vector comprises a nucleotide sequence encoding said protein of interest and growing said cell in conditions suitable for allowing the expression of the protein of interest.

The method of the invention comprises a first step of growing a microalga cell comprising a vector according to the invention, wherein the vector comprises a nucleotide sequence encoding said protein of interest. The vector of the invention may be introduced into a microalga by means of well-known techniques such as, transfection, electroporation, via particle bombardment and transformation using the vector of the invention that has been isolated. In a preferred embodiment the vector is introduced by transformation. The transformed algae may be recovered on a solid nutrient media or in liquid media.

In addition, the method of the invention comprises growing said cell in conditions suitable for allowing the expression of the protein of interest. Culture conditions suitable for the growth of the microalga and for the expression of the protein of interest may be different for each type of microalga. However, those conditions are known by skilled workers and are readily determined. In a particular embodiment, the microalga is grown under mixotrophic conditions. In a particular embodiment, the microalga is cultured in a photobioreactor in a suitable medium, under a suitable luminous intensity, at a suitable temperature. Practically any medium suitable for growing microalgae can be used; nevertheless, illustrative, non-limitative examples of said media include TAP media. The luminous intensity can vary widely, nevertheless, in a particular embodiment, the luminous intensity is comprised between 25 and 150 μmol photons m-2 s-1, particularly 100 μE. The temperature can vary usually between about 17° C. and about 30° C., particularly 25° C. The culture can be performed in the absence of aeration or with aeration. Similarly, the duration of maintenance can differ with the microalga and with the amount of protein desired to be prepared. Again, those conditions are well known and can readily be determined in specific situations. In a preferred embodiment the microalga is a green alga, more particularly from genus Chlamydomonas, and more particularly Chlamydomonas reinhardtii.

In a particular embodiment, the method of the invention further comprises purifying the protein of interest. Suitable purification can be carried out by methods known to the person skilled in the art such as by using lysis methods, extraction, ion exchange resins, electrodialysis, nanofiltration, etc.

The invention also relates to the use of a nucleotide sequence encoding a glycoprotein motif according to the invention, an expression cassette according to the invention, a vector according to the invention or a host cell according to the invention for the expression of a protein of interest.

All the terms and embodiments previously described are equally applicable to this aspect of the invention.

The invention will be described by way of the following examples which are to be considered as merely illustrative and not limitative of the scope of the invention.

### EXAMPLES

### Example 1—Identification of Endogenous Glycomodules Motifs' for Use as Fusion Sequences

Chlamydomonas most abundant secreted proteins were examined for the presence of glycomodule motifs on their mature protein sequences as disclosed in Baba et al. Plant Cell Physiol. 52, 1302-1314 (2011). Several glycomodule-like sequences were identified and tested for their effect on conferring improved recombinant protein expression (Table 1).

TABLE 1
List of selected Chlamydomonas native glycomodules. Sequences
were named according to the protein where they were found.

| Protein of | JGI | Glycomodule | |
|---|---|---|---|
| origin | protein ID | name | Sequence |
| LCL5 | 196466 | LCL | SSSPAPASSAPAPARSSSASPPPPPRRPPPRP |
| | | | (SEQ ID NO: 1) |
| GP1 | 34358 | GP1 | PPSPAPPSPAPPSPAPPSPAPPSPAPPSPGPPS |
| | | | (SEQ ID NO: 2) |
| GP2 | 195768 | GP2 | PPSPPPPTPPSPPPPSPPPPVLSPPPSPPPPSPP |
| | | | PPAPPPPSPPPPVPPPPSPPPPSPPPPSPPPAAA |
| | | | SPPPSPPPPPPPSPPPPVARLP(SEQ ID |
| | | | NO: 3) |
| PHC21 | 93464 | PHC21A | MPPPSPPPPPPPSPPPPSPPPPSPKPPSPPPPSP |
| | | | PPPSPPPPSPKPPSPKPPSPPPPSPPPP(SEQ |
| | | | ID NO: 4) |
| | | PHC21B | PPSPPPSPPPPPPPPPPPPPPPAPPPPPPP |
| | | | PPPPPPPPSPPPPPPPPPPPPPPPPPFPPP |
| | | | PPPPPPPPPSPPPP(SEQ ID NO: 5) |

### Example 2—Generation of Gene Expression Cassettes for Improved Transgene Expression

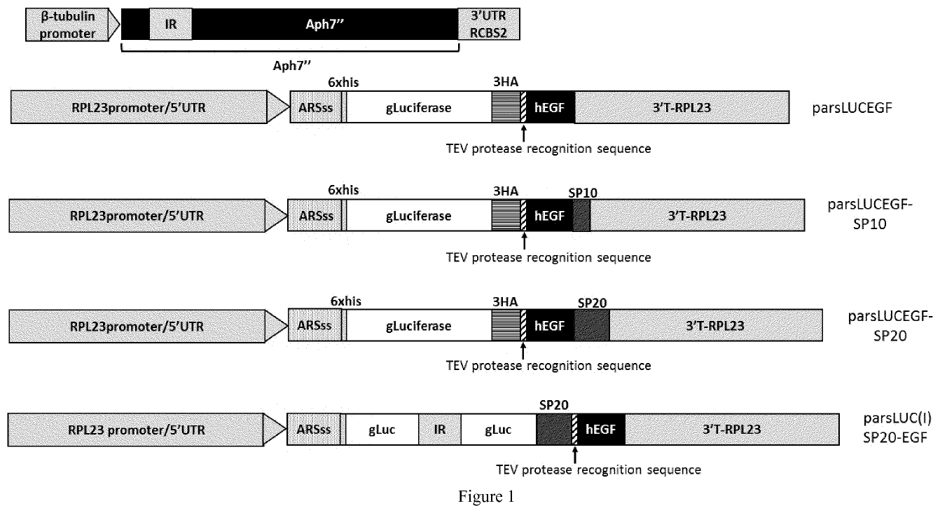

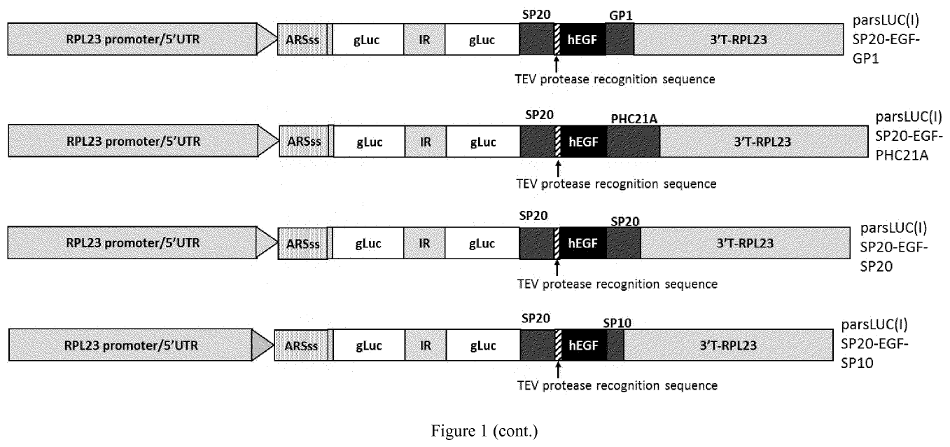

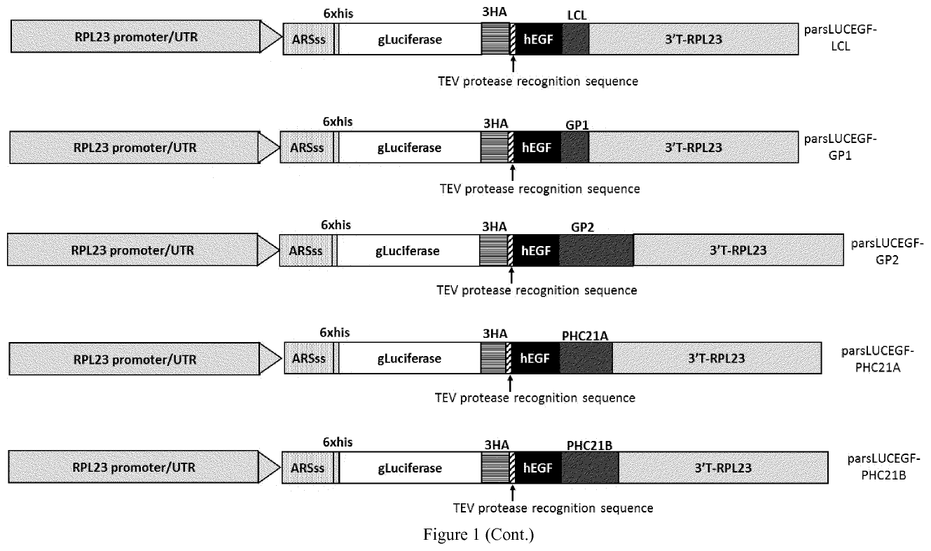

RPL23 strong constitutive promoter and regulatory regions, previously shown to surpass other commonly used promoter/UTR combinations including AR/RBCS2 described in López-Paz, C. et al., Plant J (92), 1232-1244 (2017), were used to drive expression of a gene cassette containing different elements including: ARSss secretion peptide, (SP)₁₀ or (SP)₂₀ glycomodule motifs, newly identified Chlamydomonas glycomodules LCL (SEQ ID NO: 1), GP1 (SEQ ID NO: 2) and PHC121A (SEQ ID NO: 4) (named according to original protein containing said motifs). A 6× histidine tag and 3×HA tag were added to some of these constructs (FIG. 1).

Vector containing these cassettes also contain an additional cassette that drives expression of hygromycin selectable marker ((Berthold et al. 2002. Protist 153:401-412). Genes encoding for the selectable marker may confer antibiotic resistance or gene complementation to an auxotrophic phenotype.

### Example 3—Use of Different Glycomodules Motifs' Results on Improved Recombinant Protein Expression

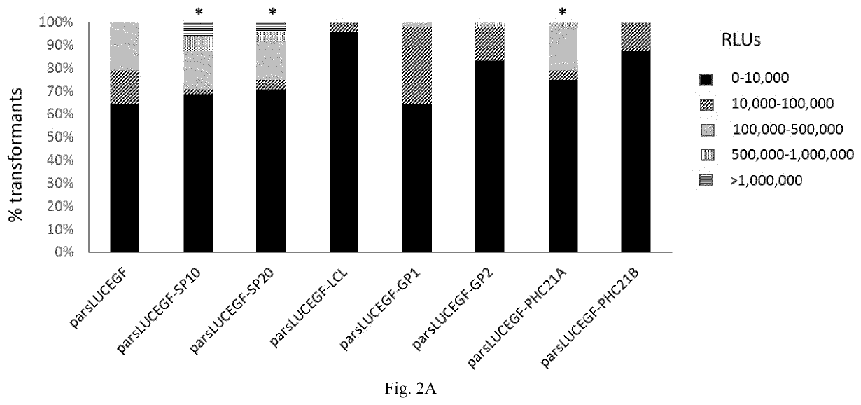

Vectors containing different cassettes as shown in FIG. 1 (plus an additional hygromycin or paromomycin cassette) were transformed into Chlamydomonas reinhardtii CC-124 and/or UVM4 strains by electroporation or glass bead transformation. After selection of transformants by growth on TAP plates containing hygromycin or paromomycin, cells were grown in 96 well plates and the effect of different glycomodules motifs on protein expression was assessed by measuring luciferase activity of recombinant fusion protein. The use of (SP)₁₀, (SP)₂₀ and PHC21A motifs (SEQ ID NO: 4) resulted in a significant increase on protein expression based on the luciferase assay (FIG. 2). Use of luciferase as a reporter protein allows to perform a high throughput screening to identify those transformants that express the highest levels of fusion recombinant protein.

### Example 4—Presence of Glycomodule Motifs' Increase Protein Stability in Media

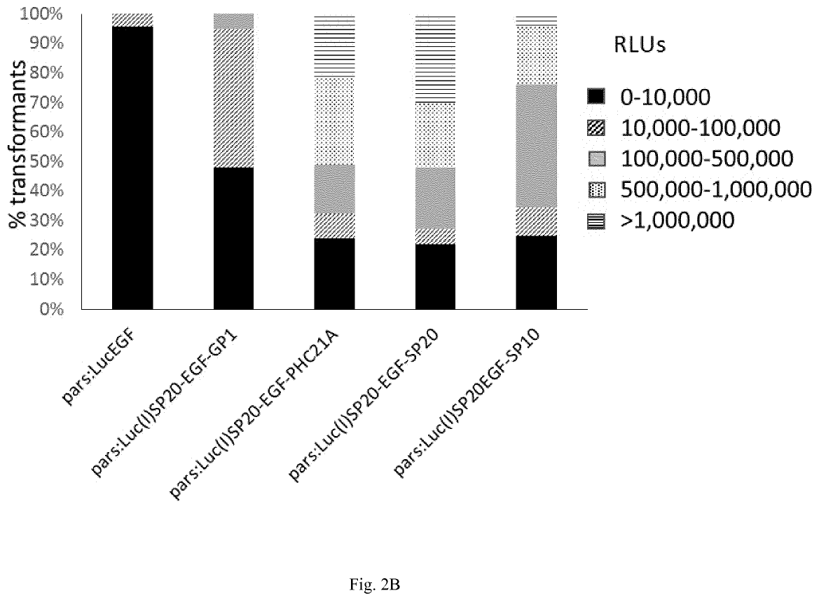

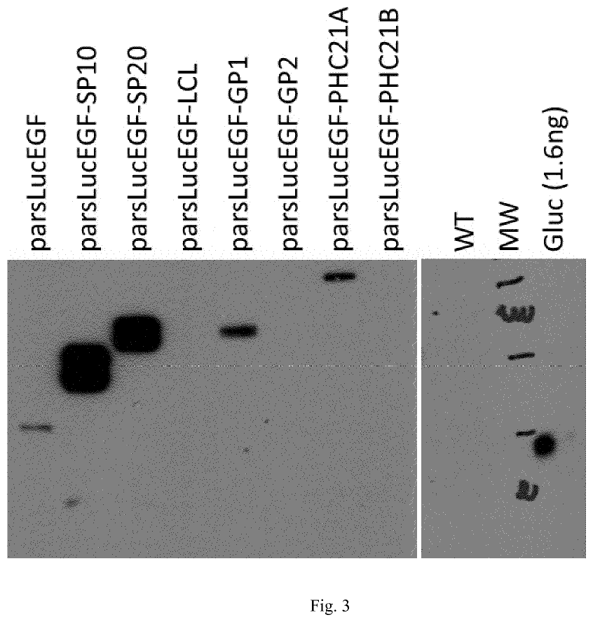

Immunoblots using anti-Gluc antibody confirmed the presence of the full-length fusion protein in the media of transformants which were selected as being those with the highest expression levels (according to luciferase assay performed in 48 independent transformants for each construct) (FIG. 3). Surprisingly, contrary to results obtained when measuring luciferase activity, transformants of construct parsLuc(I)SP20-EGF showed the highest RP levels in the media (FIG. 3). Although not seen for luciferase, parsLucEGF-GP1 also showed by western blot increased levels of protein compared to parsLucEGF (FIG. 2) GP1 construct. Although different cassettes results in different luciferase activity signal when comparing same amounts of protein (due to interference of protein of interest, tag or added glycomodules), when comparing clones transformed with same fusion protein, luciferase level correlates with the level of expressed fusion protein. Therefore, we can confirm that luciferase screening may be used as a method to detect highest expressing clones among all initially obtained transformants. Moreover, the position of the SP seems to have a positive effect on protein stability, since not only expression is increased on the construct parsLuc(I)SP20-EGF (which may be attributable to the presence of the intron) but also integrity (determined as absence of degradation). According to those results use of Chlamydomonas endogenous sequences placed between reporter gene and protein of interest (copying structure of parsLuc(I)SP20-EGF DNA cassette) may further increase protein expression and stability of said fusion protein. In fact, since more than one glycomodule motif is present at the most abundant identified secreted proteins, having more than one GM on different locations on the fusion protein may be beneficial for protein stability and, therefore, expression yield. Having different sequences, instead of repetitive sequences to introduce more than one GM, may also prevent DNA recombination during gene cassette construction, amplification or microalgae transformation. The described vector contains unique restriction sites to replace or include different GM combinations that may vary depending on nature/stability of the desired RP. It is important to note that position and type of glycomodule may affect protein biological activity and thus it is important to have more than one option available and the possibility to remove them from the final product.

### Example 5—Purification and Processing of Recombinant Secreted Proteins

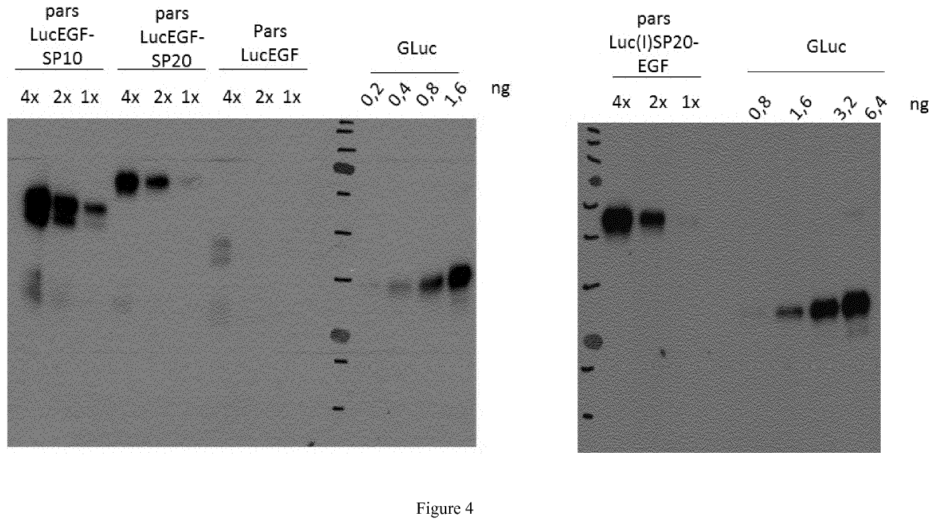

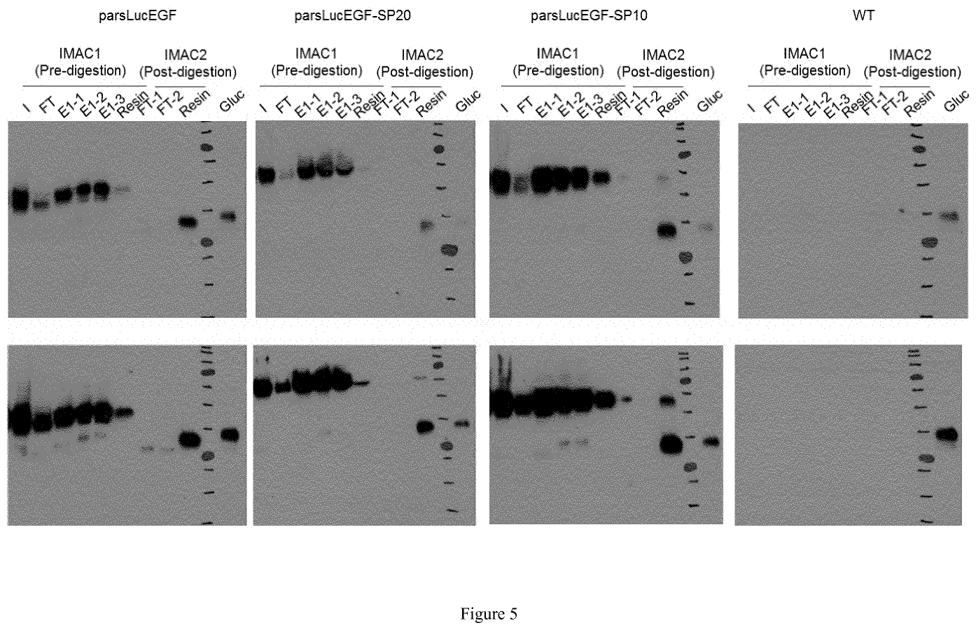

The 6× Histidine tag allowed to efficiently recover all types of secreted fusion proteins, independently of the presence of glycomodule. Media proceeding from cultures at the latest stage of growth were concentrated and applied to a nickel agarose resin. Most of the recombinant protein was bound to the resin and recovered in a single elution step (FIG. 4). After dialysis, recombinant protein was incubated with TEV protease and a second IMAC was performed to remove Gluc digested protein that remained bound to the resin. Protein not bound would contain digested protein that do not have Histidine tag (different EGF isoforms).

1. A nucleotide sequence encoding a glycomodule motif having a sequence selected from the group consisting of SEQ ID NO 1, SEQ ID NO 2, SEQ ID NO 3, SEQ ID NO 4, SEQ ID NO 5 and a functionally equivalent variant thereof, wherein the functionally equivalent variant of SEQ ID NO: 1, 2, 3, 4, or 5 has a sequence identity of at least 50% with the corresponding sequence SEQ ID NO, 1, 2, 3, 4 or 5 and wherein the sequence identity is determined throughout the whole length of the sequence SEQ ID NO: 1, 2, 3, 4 or 5.

2. (canceled)

3. An expression cassette comprising at least one nucleotide sequence encoding a glycomodule motif according to claim 1.

4. The expression cassette according to claim 3, further comprising:
(i) a nucleotide sequence encoding a secretory signal peptide,
(ii) a nucleotide sequence encoding a selectable marker,
(iii) a nucleotide sequence encoding a tag,
(iv) a regulatory nucleotide sequence,
(v) a nucleotide sequence encoding a protease recognition site,
(vi) a nucleotide sequence encoding a protein of interest or
(vii) any combination thereof.

5. The expression cassette according to claim 4, comprising from 5′ to 3′ in the same open reading frame a nucleotide sequence encoding a glycomodule motif and a nucleotide sequence encoding a protein of interest.

6. The expression cassette according to claim 5, further comprising a nucleotide sequence encoding a selectable marker in the same open reading frame as the nucleotide sequence encoding a glycomodule motif and the nucleotide sequence encoding a protein of interest.

7. The expression cassette according to claim 4, comprising a nucleotide sequence encoding a signal peptide, a nucleotide sequence encoding a first tag, a nucleotide sequence encoding a selectable marker, a nucleotide sequence encoding a second tag, a nucleotide sequence encoding a protein of interest and a nucleotide sequence encoding a glycomodule motif in the same open reading frame, wherein said open reading frame is under operation control of a regulatory nucleotide sequence.

8. The expression cassette according to claim 3, wherein a nucleotide sequence encoding a protease recognition site is located between the nucleotide sequence encoding a second tag and the nucleotide sequence encoding the protein of interest or before the nucleotide sequence encoding a glycomodule motif.

9. The expression cassette according to claim 3, comprising an additional nucleotide sequence encoding a glycomodule motif.

10. The expression cassette according to claim 9, wherein the additional nucleotide sequence encoding a glycomodule motif is selected from a nucleotide sequence encoding (SP)n and a nucleotide sequence encoding a glycomodule motif having a sequence selected from the group consisting of SEQ ID NO 1, SEQ ID NO 2, SEQ ID NO 3, SEQ ID NO 4, SEQ ID NO 5 and a functionally equivalent variant thereof, wherein the functionally equivalent variant of SEQ ID NO: 1, 2, 3, 4, or 5 has a sequence identity of at least 50% with the corresponding sequence SEQ ID NO, 1, 2, 3, 4 or 5 and wherein the sequence identity is determined throughout the whole length of the sequence SEQ ID NO: 1, 2, 3, 4 or 5.

11. An expression cassette comprising from 5″ to 3″ in the same open reading frame, a nucleotide sequence encoding a selectable marker, a nucleotide sequence encoding a glycomodule motif and a nucleotide sequence encoding a protein of interest.

12. The expression cassette according to claim 11, wherein the nucleotide sequence encoding a glycomodule motif is a nucleotide sequence encoding (SP)n, particularly (SP)10 or (SP)20.

13. The expression cassette according to claim 4, wherein the nucleotide sequence encoding a secretory signal peptide is selected from the group consisting of a nucleotide sequence encoding a secretory signal peptide from CAH, ARS or gametolysin,
wherein the nucleotide sequence encoding a selectable marker is luc (luciferase) or sh-Ble (bleomycin resistance) gene,
wherein the nucleotide sequence encoding a tag is selected from the group consisting of a nucleotide sequence encoding a hexahistidine tag and/or a 3×HA tag,
wherein the regulatory nucleotide sequence is a Chlamydomonas regulatory sequence selected from the group consisting of a sequence comprising a promoter sequence, a 5′UTR, a 3′UTR, a flanking region, an intron and any combination thereof,
wherein the regulatory sequence comprises a sequence selected from the group consisting of a HSP70A/RCBS2 chimeric promoter, the FDX gene promoter, the RPL23 gene promoter, the 3′UTR of the RCBS2 gene, the 3′UTR of the FDX gene, the 3′UTR of the RPL23 gene and any combination thereof,
wherein the nucleotide sequence encoding the protease recognition sequence encodes a TEV protease recognition sequence or a plant specific protease recognition sequence,
wherein the protein of interest is EGF,
wherein the intron is inserted into the nucleotide sequence encoding a selectable marker,
and/or wherein a nucleotide sequence encoding a linker is included between the nucleotide sequence encoding a glycomodule motif and the nucleotide sequence encoding a protease recognition sequence.

14.-22. (canceled)

23. A vector comprising a nucleotide sequence encoding a glycomodule motif according to claim 1.

24. A host cell comprising a vector according to claim 23.

25. The host cell according to claim 24, wherein the cell is Chlamydomonas reinhardtii.

26. A method for expressing a protein of interest which comprises growing a microalga cell comprising a vector according to claim 23, wherein the vector comprises a nucleotide sequence encoding a protein of interest and growing said cell in conditions suitable for allowing the expression of the protein of interest.

27. (canceled)

28. A vector comprising an expression cassette according to claim 3.

29. A host cell comprising a vector according to claim 28.


SEQUENCE LISTING (43 sequences)

SEQ ID NO: 1 - protein, 32 aa, Artificial Sequence
  glycomodule LCL
SSSPAPASSAPAPARSSSASPPPPPRRPPPRP

SEQ ID NO: 2 - protein, 33 aa, Artificial Sequence
  Glycomodule GP1
PPSPAPPSPAPPSPAPPSPAPPSPAPPSPGPPS

SEQ ID NO: 3 - protein, 90 aa, Artificial Sequence
  Glycomodule GP2
PPSPPPPTPPSPPPPSPPPPVLSPPPSPPPPSPPPPAPPPPSPPPPVPPPPSPPPPSPPPPSPPPAAASPPPSPPPPPPPSPPPPVARLP

SEQ ID NO: 4 - protein, 62 aa, Artificial Sequence
  Glycomodule PHC21A
MPPPSPPPPPPPSPPPPSPPPPSPKPPSPPPPSPPPPSPPPPSPKPPSPKPPSPPPPSPPPP

SEQ ID NO: 5 - protein, 74 aa, Artificial Sequence
  Glycomodule PHC21B
PPSPPPSPPPPPPPPPPPPPPPAPPPPPPPPPPPPPPPSPPPPPPPPPPPPPPPPPFPPPPPPPPPPPPSPPPP

SEQ ID NO: 6 - protein, 235 aa, Chlamydomonas reinhardtii
MATISSMRVGAASRVVVSGRVKTVKVAARGSWRESSTATVQASRASSATNRVSPTRSVLPANWRQELESLRNGNGSSSAASSAPAPARSSSASWRDAAPASSAPARSSSASKKAVTPSRSALPSNWKQELESLRSSSPAPASSAPAPARSSSASWRDAAPASSAPARSSSSKKAVTPSLSAPPCPLTRSWRACAAAPPPPPRRPPPRPAPRRPAGVTPPRPRRPLPAPALPPRRP

SEQ ID NO: 7 - protein, 296 aa, Chlamydomonas reinhardtii
MMRRQHAAPLVGAVNVLMVVLAFVASANAHPPSPPPSPAPPTPPTPPSPSPPSPVPPSPAPVPPSPAPPSPAPSPPPSPAPPTPSPSPSPSPSPSPSPSPSPSPSPSPSPIPSPSPKPSPSPVAVKLVWADDAIAFDDLNGTSTRPGSASRMVGEPDIAGTKCKGNLKGWMPKPSRNPRWGQAVFSGGRTVGSVANVTIRVAFATEKPALIYSSIELVVYNTGATLIRVPIAANVTRSQIRCPGFLTYGTTPIAGYPTGIDATTWPNWKIAGVRINMGAGNKKPKTSIDAVGLNLK

SEQ ID NO: 8 - protein, 1259 aa, Chlamydomonas reinhardtii
MGAVLHLAARMARSTALLVALLGLAALGAANAQGAYPPPARGLNAYCAIQNRTTNAWACNLGVNTPTACETTDLWSTDATECSLPQVPGFPVPANTFPYTDTCQYQVARLPLVYKGTTTVGGFLVLFKDYADNIYYTLSLNATARAANQPDGQWLYVEPTLPGQTGPSGAVYLWDSPPDSSKPLSVQASVQDYMIEDRNSYKRWSCATYKTSLNNYCAPGYVFDAAGICQPKAGATQGPLPSKDLSGAAGSSLFLAAEYSIVKYSVSQIGTAETGFYCGDPRTSMIAGQTLQTLALQGGVVNLAAIPGFFLPPSCKTTSRPPSPPLPPSPPPQPPSPLPPSPAPLPPSPPPSPLPPSPKPPTPPSPLPPAPPTQQFTGSVFVYAPNRPNGFNQDDVTKVYAMMRYGLGCGIGNNPNVCLKSQPYVTINETNNAFYPAYTMLGVKMFFDGSKDGSGQPYPTSEQNKWAADAVTSMFAVLGSSELWKALAGPVQFGGLGLGCGAFMSFEAVVDGQYYSQPIGGGSPPLVDSYDGSTYTYQGARYVCPLLLGGFGGPQWPNASLPYYIPDGVAGLPGYIRLGAIPQNTNYQRRRCATILTPPDLMQPSTSCPLSPPPSPSPPPSPPQPPSPPPVPPSPPSPPPSPPSPANPSPPPPAPPAAFCGLNFKVISRIPKNGVDRNPDCSLLATLVQSTFTQGVQLRNPPQFRCGTTVSATEISAVADTLTTEGAQVFLRNMAGNIGAFNQFTFPAPGGLGLICGDYFNVTSTCGMNTTAPVPVFQPNGTVVNATYPFVVGPDAIFYPQFVLPSYTCPPPPPAPSPPPKPPTPSPPPLPPQPNPPPAPPSPNPSPPPPPPGFRFQMSVINGDQNDAINCPRYTSWMNAMMDSFERTNTIQRVNPSAPYCSRPAQETLLSPELAQPSQVNFLYQYLSVNSTIGVFVRDGGVPCGSAVRLYNPAGGGFFTDYRCSRDVPTNPAVAVLDLCCPLPPSPPPPTPPSPPPPSPPPPVLSPPPSPPPPSPPPPAPPPPSPPPPVPPPPSPPPPSPPPPSPPPAAASPPPSPPPPPPPSPPPPVARLPPWPPLPVNNVPPAPTARNTTWTAPAGTTVRTYEIFAPFKGTGGRNTGVSGRTVQFWLCPALKAALQDQLGQPVSVKPNVNASISCTDWYQVKKKAKGLFYRITVAAQQSAHDAVMDFILSGGDFVRQARLVCGSQIRIGEAEPPRQKLKAVFPSPFDPKAAVDPYLYLSGKAGSPGAGVCIDKLPV

SEQ ID NO: 9 - protein, 192 aa, Chlamydomonas reinhardtii
MRSIGYKSQGARAPKCDPRLANRTPYSLSFKSKSSRTVNGFPANVYCFSIKVDAGVCGNSRCCNMDLDKVEWLTDTTNCRQAAVGYTVSIRPTDVKMPVWTRQIDNTVVPATSFDVYKTNQLGLTLGNADGAEICLILKQKSSCPTLDTFCSQGDIGCQYAVFDKTQNCCAEDWSLNAGDGSFSRRRFRLAL

SEQ ID NO: 10 - DNA, 60 bp, Artificial Sequence
  Nucleotide sequence encoding the signal peptide from CAH1 Chlamydomonas reinhardtii carbonic anhydrase (CAH1)
atggcgcgtactggcgctctactcctggtcgcgctggcgcttgcgggctgcgcgcaggct

SEQ ID NO: 11 - protein, 20 aa, Artificial Sequence
  signal peptide from CAH1
MARTGALLLVALALAGCAQA

SEQ ID NO: 12 - DNA, 90 bp, Artificial Sequence
  nucleotide sequence encoding the signal peptide from ARS1, Chlamydomonas reinhardtii periplasmic arylsulfatase (ARS1)
atgcatgcacgcaagatgggtgccctcgcggtgctcgccgtcgcttgcctcgcggcagtggcatcggttgcgcatgcggccgacaccaaa

SEQ ID NO: 13 - protein, 30 aa, Artificial Sequence
  signal peptide from ARS
MHARKMGALAVLAVACLAAVASVAHAADTK

SEQ ID NO: 14 - DNA, 84 bp, Artificial Sequence
  nucleotide sequence encoding the signal peptide from Gametolysin M11, Chlamydomonas reinhardtii
atgtcgctggcgacgcggcgcttcggcgccgcagcggcgcttctagtcgccgcatgcgtgctgtgcacagctcctgcgtgggcc

SEQ ID NO: 15 - protein, 28 aa, Artificial Sequence
  signal peptide from gametolysin
MSLATRRFGAAAALLVAACVLCTAPAWA

SEQ ID NO: 16 - DNA, 392 bp, Artificial Sequence
  ble resistance sequence
atggccatgcacatggccaagctgaccagcgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccagaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 17 - DNA, 909 bp, Artificial Sequence
  RPL23 promoter
ctctagagcacgtccataatgaaaggtccgtgactcatgaagccagcacacacgtgatgatatcgttccgctgtggctggggtggagccttcgtcaatacggtaacaacgctagctatgatgccggagcccggaggaatgacgggatgtacaaggttgaaccgctggtgaggtagcagccttcaggcctagctgcatgcacccagacgttgttgtgtgtaagcctctgttcgtgctttactgcttccaagactctctcgctacagctgcgtgaaacgaaagatagtcgatgcgcagcttggggaagtcctcgtgctgcttgcgcaagctccaattccgcctccctcctcccaccctcacactactcccgcacaccccatccccacacaccctgctccccttaacaccgactgcacctcccctccggccccctccgcccccctccggccccctgcaacactatccctccgcctcccttcggcgcagtctcccgttgtggccaaccgcgggcgcaaagctgaacctcaccttaatcctggtgctccacacctgtgccacctacgccctgtcacagggtgaagataaagccatggcatctcccgtcgattatttggctgccgtttcttggctgccgcttctccgaccgctacacagggtgcccgccaggcgtggttcaagtgccagcggggctgccagcgctctcgccgggttcgtgccgagttgaagcaacctccctgagcctcctcgcggcctgcaaatcagtattgataataccaatacagtaggcatattcttcaagcctgctagcggaactagtaaggccctctgcgcagaagttcggcaaaggctccagggcccattccgtctctctcccgagcgagctttgacgcgcggcccgcgactgtttctt

SEQ ID NO: 18 - DNA, 687 bp, Artificial Sequence
  ferredoxin 1 FDX promoter
ggtacctggaggggtaatccgtgggggtaatcgcagggacccgcaaaccccccgtctgcgccgaacttccgcggcatatccctggcgaccccaatcgcgacccaccaactggcctgcatgatgatactgtgcatgcatgctggggctcgcacgccgtccgaacccgcgcgcaaagctggcccgcggccacgaaatcgtggcatttgcacttgccggtccgattctactatgccgcgccacgagcagcgacgcgcgaagcccatttcccctgtcatacaaattcaggaagcccatggtcacacattagagccgcgcatgtaggtcaagctgtctgcgtagggtcctgtttgcgtggctttggcatgtgcacccggcgcccagcgcccgagcgcaattcaagtggttaagacgcctcagaacgcgtaattgaatgcatgtgtcattctggaagggtgtatagagcggtcgagcggaaggctgtccagctcgcgtgcaagcaattgtctcgcgacgcccgctcccgacgagtgcgaccgcccgcgaccgggttcgctggtgcaagtggccaatgcgagcgctgggcgcagagaaaaaggcgacgtacggcgatgaaggctccagggcggatgggcgaggggcaaccgggataaatggtcctcctttccccaaggcccgctacactcttga

SEQ ID NO: 19 - DNA, 473 bp, Artificial Sequence
  HSP70-RCBS2 chimeric promoter, also known as AR
tcgctgaggcttgacatgattggtgcgtatgtttgtatgaagctacaggactgatttggcgggctatgagggcgggggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaa

SEQ ID NO: 20 - DNA, 193 bp, Artificial Sequence
  3′UTR of RPL23
catatgtaaaggtgaggcgggggttcggagggactaggggaagagttggacgccagagtggggcgggttttcgggcctctgacatgcgcgcgtgccttcccaagcacaactcaccatctgactcgcatgctgacggtcctctgtgtcccttggcccttgcgcgcagattctgcacgtgtaacggtccgcctgt

SEQ ID NO: 21 - DNA, 179 bp, Artificial Sequence
  3′UTR of the RCBS2 gene
atggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgatt

SEQ ID NO: 22 - DNA, 202 bp, Artificial Sequence
  3′UTR of the FDX gene promoter
ggatccggcctcgaggcgctttcgccatctgcgggggtcgtaggctagaactggggttggggatcgggctgcttgcatagccaagcaattttccatctggccagcaatggcctagcactatgagcggttcaagtgtctcttgtgtgttgtgtcgcattgcatggccgtggtgacctgcaattttctgtaaccggacatgcag

SEQ ID NO: 23 - DNA, 541 bp, Artificial Sequence
  terminator and flanking region of 3′FDX
aagcttcggttcgcgtcctttcctcgcttgctacgggatcgggagtcggcagggctagaagacttgggtaacacgcgcaattcagcaatacaggccagccagggcagcgaagggggacttcagcaaggacctctcgggaatagtggaagagctaggaggggtacagcagtagagatcgaggggtccagctcacagttctattacgtcgttcgtggggacgaactgggtcgaggcgctacggactcgaaatgacggcagagggtggcaaggaaggggagcaccactgtgagcagttgcagcggcacatacactacgtctcttggccttaagcacagccagcacacttgtacggggcaacagtagccccgaagcagcctgatgcagtcacaccgtgccgggccagtgttaacaaggaagggcaggcaccagggcgagggcaggcgcggcaaaactcgccggttcctgacacggtgacacgcaggtatacggtgacagctcagctagtgataccagctgctccgcgttctgaggagaggagctc

SEQ ID NO: 24 - DNA, 146 bp, Artificial Sequence
  RCBS2 intron sequence
gtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcagg

SEQ ID NO: 25 - DNA, 21 bp, Artificial Sequence
  nucleotide sequence coding TEV protease sequence
gagaacctgtacttccagagc

SEQ ID NO: 26 - protein, 5 aa, Artificial Sequence
  Linker repeated n times
GGGGS

SEQ ID NO: 27 - protein, 20 aa, Artificial Sequence
  (SP)10
SPSPSPSPSPSPSPSPSPSP

SEQ ID NO: 28 - protein, 40 aa, Artificial Sequence
  (SP)20
SPSPSPSPSPSPSPSPSPSPSPSPSPSPSPSPSPSPSPSP

SEQ ID NO: 29 - DNA, 109 bp, Artificial Sequence
  5′UTR- RPL23
tctctcaccgaggtggtcagcaggtaggcaaattgcattttcttttattcatttaggtccttgagaactccgctcacagctttctcttgtctgcgcgcagagtgaattc

SEQ ID NO: 30 - DNA, 69 bp, Artificial Sequence
  5′UTR- FDX
ccttgctcacgtagtgtccttcgtcgccactcacaaccttcaaaagaattcggcagatctggcggatcc

SEQ ID NO: 31 - DNA, 23 bp, Artificial Sequence
  5′UTR RBCS2
gagaagtcactcaacatcttaaa

SEQ ID NO: 32 - DNA, 545 bp, Artificial Sequence
  3′terminator and flanking regions RPL23
gagctttccggtcccgaccctcgctcccgttcatcgcgctcggcgcggcagcagttgtagcggacagcagcagcggcagcaggggccctgccagcacgaaaggagtagcagcctggggcggacactgtcagcagagccggcagccggactgtttgaggttactcagtgccaattgcagcatttgagctgcctccaggcagcggcggcagcagctggaaagtgtggccagtggcggtttggatcagcatgactgtgacgtgctagataacagcagcggcaggcaatgagcatgcagaatagcgaggaaccgggagggttgtgaggagtggacgaaggggcaagcggctctgcccatcgcgagcgaacccgcaacagcgagggcactcagcccgtaacgcgcaagtgccctccaccaaacagcagcagcagcaagaaacgcaaacagctgggagcatgtctatttctgcgctgtggcattgcaggtgcttgccttggacccgtgcggagggagcatacgtctgcggcgaagctgcgcctggggtacc

SEQ ID NO: 33 - DNA, 42 bp, Artificial Sequence
  3′flanking region RBCS2
ttggcctctttctccatgggcgggctgggcgtatttgaagcg

SEQ ID NO: 34 - DNA, 162 bp, Homo sapiens
aacagcgacagcgagtgccccctgagccacgacggctactgcctgcacgacggcgtgtgcatgtacatcgaggccctggacaagtacgcctgcaactgcgtcgtgggctacatcggcgagcgctgccagtaccgcgacctgaagtggtgggagctgcgctac

SEQ ID NO: 35 - DNA, 513 bp, Artificial Sequence
  luciferase
aagcccaccgagaacaacgaggacttcaacatcgtggccgtggccagcaacttcgccaccaccgacctggacgccgaccgcggcaagctgcccggcaagaagctgcccctggaggtgctgaaggagatggaggccaacgcccgcaaggccggctgcacccgcggctgcctgatctgcctgagccacatcaagtgcacccccaagatgaagaagttcatccccggccgctgccacacctacgagggcgacaaggagagcgcccagggcggcatcggcgaggccatcgtggacatccccgagatccccggcttcaaggacctggagcccatggagcagttcatcgcccaggtggacctgtgcgtggactgcaccaccggctgcctgaagggcctggccaacgtgcagtgcagcgacctgctgaagaagtggctgccccagcgctgcgccaccttcgccagcaagatccagggccaggtggacaagatcaagggcgccggcggcgacgatacgggc

SEQ ID NO: 36 - DNA, 538 bp, Artificial Sequence
  sh-Ble marker(contains RBCS2 intron)
atggccatgcacatggccaagctgaccagcgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactga

SEQ ID NO: 37 - DNA, 1018 bp, Artificial Sequence
  RPL23 promoter + 5′UTR
ctctagagcacgtccataatgaaaggtccgtgactcatgaagccagcacacacgtgatgatatcgttccgctgtggctggggtggagccttcgtcaatacggtaacaacgctagctatgatgccggagcccggaggaatgacgggatgtacaaggttgaaccgctggtgaggtagcagccttcaggcctagctgcatgcacccagacgttgttgtgtgtaagcctctgttcgtgctttactgcttccaagactctctcgctacagctgcgtgaaacgaaagatagtcgatgcgcagcttggggaagtcctcgtgctgcttgcgcaagctccaattccgcctccctcctcccaccctcacactactcccgcacaccccatccccacacaccctgctccccttaacaccgactgcacctcccctccggccccctccgcccccctccggccccctgcaacactatccctccgcctcccttcggcgcagtctcccgttgtggccaaccgcgggcgcaaagctgaacctcaccttaatcctggtgctccacacctgtgccacctacgccctgtcacagggtgaagataaagccatggcatctcccgtcgattatttggctgccgtttcttggctgccgcttctccgaccgctacacagggtgcccgccaggcgtggttcaagtgccagcggggctgccagcgctctcgccgggttcgtgccgagttgaagcaacctccctgagcctcctcgcggcctgcaaatcagtattgataataccaatacagtaggcatattcttcaagcctgctagcggaactagtaaggccctctgcgcagaagttcggcaaaggctccagggcccattccgtctctctcccgagcgagctttgacgcgcggcccgcgactgtttctttctctcaccgaggtggtcagcaggtaggcaaattgcattttcttttattcatttaggtccttgagaactccgctcacagctttctcttgtctgcgcgcagagtgaattc

SEQ ID NO: 38 - DNA, 738 bp, Artificial Sequence
  3′UTR RPL23 containing 3′UTR/ terminator+flanking region
catatgtaaaggtgaggcgggggttcggagggactaggggaagagttggacgccagagtggggcgggttttcgggcctctgacatgcgcgcgtgccttcccaagcacaactcaccatctgactcgcatgctgacggtcctctgtgtcccttggcccttgcgcgcagattctgcacgtgtaacggtccgcctgtgagctttccggtcccgaccctcgctcccgttcatcgcgctcggcgcggcagcagttgtagcggacagcagcagcggcagcaggggccctgccagcacgaaaggagtagcagcctggggcggacactgtcagcagagccggcagccggactgtttgaggttactcagtgccaattgcagcatttgagctgcctccaggcagcggcggcagcagctggaaagtgtggccagtggcggtttggatcagcatgactgtgacgtgctagataacagcagcggcaggcaatgagcatgcagaatagcgaggaaccgggagggttgtgaggagtggacgaaggggcaagcggctctgcccatcgcgagcgaacccgcaacagcgagggcactcagcccgtaacgcgcaagtgccctccaccaaacagcagcagcagcaagaaacgcaaacagctgggagcatgtctatttctgcgctgtggcattgcaggtgcttgccttggacccgtgcggagggagcatacgtctgcggcgaagctgcgcctggggtacc

SEQ ID NO: 39 - DNA, 756 bp, Artificial Sequence
  FDX promoter + 5′UTR
ggtacctggaggggtaatccgtgggggtaatcgcagggacccgcaaaccccccgtctgcgccgaacttccgcggcatatccctggcgaccccaatcgcgacccaccaactggcctgcatgatgatactgtgcatgcatgctggggctcgcacgccgtccgaacccgcgcgcaaagctggcccgcggccacgaaatcgtggcatttgcacttgccggtccgattctactatgccgcgccacgagcagcgacgcgcgaagcccatttcccctgtcatacaaattcaggaagcccatggtcacacattagagccgcgcatgtaggtcaagctgtctgcgtagggtcctgtttgcgtggctttggcatgtgcacccggcgcccagcgcccgagcgcaattcaagtggttaagacgcctcagaacgcgtaattgaatgcatgtgtcattctggaagggtgtatagagcggtcgagcggaaggctgtccagctcgcgtgcaagcaattgtctcgcgacgcccgctcccgacgagtgcgaccgcccgcgaccgggttcgctggtgcaagtggccaatgcgagcgctgggcgcagagaaaaaggcgacgtacggcgatgaaggctccagggcggatgggcgaggggcaaccgggataaatggtcctcctttccccaaggcccgctacactcttgaccttgctcacgtagtgtccttcgtcgccactcacaaccttcaaaagaattcggcagatctggcggatcc

SEQ ID NO: 40 - DNA, 743 bp, Artificial Sequence
  3′UTR FDX containing 3′UTR/terminator+flanking region
ggatccggcctcgaggcgctttcgccatctgcgggggtcgtaggctagaactggggttggggatcgggctgcttgcatagccaagcaattttccatctggccagcaatggcctagcactatgagcggttcaagtgtctcttgtgtgttgtgtcgcattgcatggccgtggtgacctgcaattttctgtaaccggacatgcagaagcttcggttcgcgtcctttcctcgcttgctacgggatcgggagtcggcagggctagaagacttgggtaacacgcgcaattcagcaatacaggccagccagggcagcgaagggggacttcagcaaggacctctcgggaatagtggaagagctaggaggggtacagcagtagagatcgaggggtccagctcacagttctattacgtcgttcgtggggacgaactgggtcgaggcgctacggactcgaaatgacggcagagggtggcaaggaaggggagcaccactgtgagcagttgcagcggcacatacactacgtctcttggccttaagcacagccagcacacttgtacggggcaacagtagccccgaagcagcctgatgcagtcacaccgtgccgggccagtgttaacaaggaagggcaggcaccagggcgagggcaggcgcggcaaaactcgccggttcctgacacggtgacacgcaggtatacggtgacagctcagctagtgataccagctgctccgcgttctgaggagaggagctc

SEQ ID NO: 41 - DNA, 650 bp, Artificial Sequence
  AR promoter +5′UTR
tcgctgaggcttgacatgattggtgcgtatgtttgtatgaagctacaggactgatttggcgggctatgagggcgggggaagctctggaagggccgcgatggggcgcgcggcgtccagaaggcgccatacggcccgctggcggcacccatccggtataaaagcccgcgaccccgaacggtgacctccactttcagcgacaaacgagcacttatacatacgcgactattctgccgctatacataaccactcagctagcttaagatcccatcaagcttgcatgccgggcgcgccagaaggagcgcagccaaaccaggatgatgtttgatggggtatttgagcacttgcaacccttatccggaagccccctggcccacaaaggctaggcgccaatgcaagcagttcgcatgcagcccctggagcggtgccctcctgataaaccggccagggggcctatgttctttacttttttacaagagaagtcactcaacatcttaaaatggccaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcagg

SEQ ID NO: 42 - DNA, 221 bp, Artificial Sequence
  3′UTR RCB2 containing 3′UTR/terminator+flanking region
atggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcg

SEQ ID NO: 43 - DNA, 1717 bp, Artificial Sequence
  APH7 cassette
gaattcgatatcaagcttctttcttgcgctatgacacttccagcaaaaggtagggcgggctgcgagacggcttcccggcgctgcatgcaacaccgatgatgcttcgaccccccgaagctccttcggggctgcatgggcgctccgatgccgctccagggcgagcgctgtttaaatagccaggcccccgattgcaaagacattatagcgagctaccaaagccatattcaaacacctagatcactaccacttctacacaggccactcgagcttgtgatcgcactccgctaagggggcgcctcttcctcttcgtttcagtcacaacccgcaaacatgacacaagaatccctgttacttctcgaccgtattgattcggatgattcctacgcgagcctgcggaacgaccaggaattctgggaggtgagtcgacgagcaagcccggcggatcaggcagcgtgcttgcagatttgacttgcaacgcccgcattgtgtcgacgaaggcttttggctcctctgtcgctgtctcaagcagcatctaaccctgcgtcgccgtttccatttgcagccgctggcccgccgagccctggaggagctcgggctgccggtgccgccggtgctgcgggtgcccggcgagagcaccaaccccgtactggtcggcgagcccggcccggtgatcaagctgttcggcgagcactggtgcggtccggagagcctcgcgtcggagtcggaggcgtacgcggtcctggcggacgccccggtgccggtgccccgcctcctcggccgcggcgagctgcggcccggcaccggagcctggccgtggccctacctggtgatgagccggatgaccggcaccacctggcggtccgcgatggacggcacgaccgaccggaacgcgctgctcgccctggcccgcgaactcggccgggtgctcggccggctgcacagggtgccgctgaccgggaacaccgtgctcaccccccattccgaggtcttcccggaactgctgcgggaacgccgcgcggcgaccgtcgaggaccaccgcgggtggggctacctctcgccccggctgctggaccgcctggaggactggctgccggacgtggacacgctgctggccggccgcgaaccccggttcgtccacggcgacctgcacgggaccaacatcttcgtggacctggccgcgaccgaggtcaccgggatcgtcgacttcaccgacgtctatgcgggagactcccgctacagcctggtgcaactgcatctcaacgccttccggggcgaccgcgagatcctggccgcgctgctcgacggggcgcagtggaagcggaccgaggacttcgcccgcgaactgctcgccttcaccttcctgcacgacttcgaggtgttcgaggagaccccgctggatctctccggcttcaccgatccggaggaactggcgcagttcctctgggggccgccggacaccgcccccggcgcctgataaggatccccgctccgtgtaaatggaggcgctcgttgatctgagccttgccccctgacgaacggcggtggatggaagatactgctctcaagtgctgaagcggtagcttagctccccgtttcgtgctgatcagtctttttcaacacgtaaaaagcggaggagttttgcaattttgttggttgtaacgatcctccgttgattttggcctctttctccatgggcgggctgggcgtatttgaagcg